(19) United States
(12) Patent Application Publication
(10) Pub. No.: US 2025/0375436 A1
(43) Pub. Date: Dec. 11, 2025

(54) SERDEXMETHYLPHENIDATE FOR TREATMENT OF IDIOPATHIC HYPERSOMNIA

(71) Applicant: ZEVRA THERAPEUTICS, INC., Celebration, FL (US)

(72) Inventors: Sven Guenther, Coralville, IA (US); Travis Mickle, Kissimmee, FL (US); Rene Braeckman, Celebration, FL (US); Guochen Chi, Coralville, IA (US)

(21) Appl. No.: 19/088,541

(22) Filed: Mar. 24, 2025

Related U.S. Application Data
(60) Provisional application No. 63/569,724, filed on Mar. 25, 2024.
(60) Provisional application No. 63/654,915, filed on May 31, 2024.
(60) Provisional application No. 63/654,960, filed on Jun. 1, 2024.
(60) Provisional application No. 63/676,353, filed on Jul. 27, 2024.
(60) Provisional application No. 63/691,651, filed on Sep. 6, 2024.
(60) Provisional application No. 63/712,865, filed on Oct. 28, 2024.
(60) Provisional application No. 63/712,861, filed on Oct. 28, 2024.

(51) Int. Cl. A61K 31/4545; A61P 25/00

(52) U.S. Cl. CPC A61K 31/4545; A61P 25/00

(57) ABSTRACT

The present technology relates to a method of treating sleep disorders in a patient, the method comprising: administering to the patient before bedtime a composition comprising a therapeutically effective amount of a conjugate of d-methylphenidate having a structure of Formula I or a pharmaceutically acceptable salt thereof, wherein the composition comprising the conjugate of d-methylphenidate is administered about 0-3 hours before the bedtime of the patient.

### RELATED APPLICATIONS

This application claims priority to U.S. Provisional Applications 63/712,865, filed on Oct. 28, 2024; 63/712,861; filed on Oct. 28, 2024; 63/691,651, filed on Sep. 6, 2024; 63/676,353, filed on Jul. 27, 2024; 63/654,960, filed on Jun. 1, 2024; 63/654,915, filed on May 31, 2024; and 63/569,724, filed on Mar. 25, 2024, the entire contents of which are incorporated by reference herein.

### FEDERALLY SPONSORED RESEARCH OR DEVELOPMENT

Not Applicable

### BACKGROUND OF THE INVENTION

Idiopathic hypersomnia (IH) is a rare sleep disorder characterized by excessive daytime sleepiness. Patients with IH experience daytime lapses into sleep, or an irrepressible need to sleep that persists even with adequate or prolonged nighttime sleep. Additionally, those with IH may have difficulty awakening or getting out of bed in the morning, otherwise known as “sleep inertia”. IH patients may also show symptoms of “brain fog”, and often fall asleep unintentionally or at inappropriate times. These symptoms of IH often lead to further, even more debilitating problems such as memory lapses, difficulty maintaining focus, and depression.

It is estimated, based on claims data, that approximately 37,000 patients in the United States are currently diagnosed with IH, although the total patient population may be larger due to some patients who have not yet been diagnosed, have been misdiagnosed, or are not currently seeking treatment.

Conventionally, sleep disorders like IH are treated by administering several classes of drugs, including depressants, such as sodium oxybates, to induce sleep. However, such drugs can make it difficult for a patient with IH associated with Excessive Daytime Sleepiness (EDS) and/or Sleep Inertia (SI) to reach a full state of wakefulness following sleep. Additionally, these drugs have complicated dosing regimens, often requiring a second nightly dose that interrupts the patient's sleep patterns. Oxybates also carry a risk developing drug dependence.

These issues result in a high percentage of patients to seek second line treatments, including Central Nervous System (CNS) stimulants. However, patients prescribed conventional CNS stimulants such as modafinil or amphetamine continue to struggle with Sleep Inertia and Brain Fog.

Methylphenidate (“MPH”), are believed to enhance the activity of the sympathetic nervous system and/or (CNS). Stimulants such as MPH and the various forms and derivatives thereof are used for the treatment of a range of conditions and disorders predominantly encompassing, for example, sleep disorders, including, but not limited to narcolepsy, excessive daytime sleepiness, central hypersomnolence disorders and/or wakefulness. Methylphenidate is a psychostimulant which is a chain substituted amphetamine derivative. Similar to amphetamine and cocaine, methylphenidate targets the central nervous system, specifically the dopamine transporter (DAT) and norepinephrine transporter (NET). Methylphenidate is thought to act by increasing the concentrations of dopamine and norepinephrine in the synaptic cleft by inhibiting their reuptake into the axon terminal at both the dopamine transporter (DAT) and the norepinephrine transporter (NET).

Although an amphetamine derivative, the pharmacology of methylphenidate and amphetamine differ, as amphetamine is also a dopamine transport substrate that actively promotes release of dopamine into the synaptic cleft whereas methylphenidate functions as a dopamine transport blocker. As a norepinephrine and dopamine re-uptake inhibitor, methylphenidate thus blocks re-uptake of dopamine and norepinephrine (noradrenaline) into presynaptic neurons (and possibly stimulates the release of dopamine from dopamine nerve terminals at high doses), thereby increasing the levels of dopamine and norepinephrine in the synapse. In some in vitro studies, methylphenidate has been shown to be more potent as an inhibitor of norepinephrine uptake/re-uptake when compared to dopamine. However, some in vivo studies have indicated that methylphenidate is more potent in potentiating extracellular dopamine concentrations than norepinephrine concentrations. Unlike amphetamine, it has been suggested in the scientific and/or clinical research community that methylphenidate does not seem to significantly facilitate the release of these two monoamine neurotransmitters into the synaptic cleft at therapeutic doses.

Four isomers of methylphenidate are known to exist: d-erythro-methylphenidate, l-erythro-methylphenidate, d-threo-methylphenidate, and 1-threo-methylphenidate. Originally, methylphenidate was marketed as a mixture of two racemates, d/l-erythro-methylphenidate and d/l-threo-methylphenidate. Subsequent research showed that most of the desired pharmacological activity of the mixture is associated with the threo-isomer resulting in the marketing of the isolated threo-methylphenidate racemate. Later, the scientific community determined that the d-threo-isomer is primarily responsible for the stimulant activity. Consequently, new products were developed containing only d-threo-methylphenidate (also known as “d-threo-MPH” or “dexmethylphenidate” or “d-MPH”).

U.S. Pat. No. 11,505,537 discloses conjugates of d-methylphenidate for treating certain sleep disorders such as central hypersomnolence disorders, idiopathic hypersomnia, obstructive sleep apnea, or shift work disorders.

However there remains a need in the art for forms of d-methylphenidate that can provide treatment for Excessive Daytime Sleepiness, Brain Fog, and/or Sleep Inertia associated with Idiopathic Hypersomnia through a simple dosing regimen. Additionally, there remains a need in the art for a treatment of Brain Fog and/or Sleep Inertia not associated with IH. Furthermore, there remains a need in the art for a treatment of Brain Fog and/or Sleep Inertia associated with IH, that does not require a second-line medication. Likewise in this regard, there remains a need in the art for a once-daily treatment regimen for these conditions not associated with IH that does not require either a second nightly dose of the medication nor requires a second line regiment of treatment.

Moreover, there remains a need in the art for methods of treatment of Idiopathic Hypersomnia, Excessive Daytime Sleepiness, Brain Fog, and/or Sleep Inertia without serious and/or adverse side effects associated with certain current treatment regimens and modalities not employing conjugates of d-methylphenidate. At present, certain methods of treatment for sleep disorders such as Excessive Daytime Sleepiness utilize a depressant, such as sodium oxybate, that carry “black-box” warnings of serious side-effects, including death, when used in combination with other Central Nervous System (CNS) depressants such as general anesthetics, alcohol and opioid analgesics that can be found, for instance, in common night time medicines. In addition, many current treatment regimens involve at least two medications to address such sleep disorders, compounding the risk of side effects not just from the drugs/medicaments singularly, but also from the undisclosed risk of compounding side-effects and undisclosed risk of new side-effects from the combination of said medications/medicaments/treatment modalities.

Patients with IH suffer from a myriad of debilitating symptoms that are not limited to excessive daytime sleepiness. In the pivotal study for oxybate, although mean improvement relative to placebo were evidenced across several endpoints, 21% of oxybate-treated patients reported worsening symptoms (as assessed on the Patient Global Impression of Change [PGI-C]scale), indicative of a lack of efficacy in this subset. In addition, oxybate is associated with certain treatment-emergent adverse events (TEAEs), including nausea (22% of subjects), headache (18%), dizziness (12%), anxiety (11%), and vomiting (11%), some of which lead to discontinuing the medication (Dauvilliers 2022). As well, the prevalence of sleep inertia remains high despite treatment with Xywav (Bae 2023) and impact on brain fog has not been reported.

Because oxybate is a central nervous system (CNS) depressant that can cause respiratory depression, it is contraindicated in combination with alcohol and sedative hypnotics.

While there are studies of varying quality demonstrating that alerting agents and oxybate can ameliorate EDS in patients with IH, the response to treatment is frequently incomplete and some patients experience dose-limiting adverse effects that are typical of alerting agents and oxybate (Ali 2009, Thakrar 2018, Arnulf 2023). Oxybate showed evidence of improvement on measures of EDS, sleep inertia, and certain consequences of IH (Dauvilliers 2022). However, it has been found to lead to increases in depressive symptoms in some patients (Arnulf 2023). Cognitive deficits described as “brain fog” are pervasive in patients with narcolepsy and IH (Trotti 2017, Rosenberg 2024). As a CNS depressant, it is unlikely that oxybate can directly improve brain fog and would be predicted to worsen this condition in some patients. Evidence suggests that satisfaction with treatment would be enhanced if, in addition to EDS, other core symptoms (e.g., cognitive impairment, brain fog) were treated (CDER 2014, Schneider 2023, Stevens 2023, Arnulf 2023, Rosenberg 2024). Accordingly, there remains a need for additional efficacious treatments that can not only address EDS, but potentially other symptoms that are associated with poor quality of life in patients with narcolepsy and IH (Arnulf 2023, Rosenberg 2024).

There remains further need in the art for forms of d-methylphenidate that can provide treatment for these disorders that persists throughout the day. With respect to the historical use of stimulants such as MPH for treating IH, suboptimal efficacy and dose-limiting adverse effects may be due, in part, to the pharmacokinetic (PK) profile of specific formulations employed during treatment. For example, it is common for immediate-release MPH to be administered 3-4 times per day when used to treat excessive daytime sleepiness (Ali 2009). The resulting PK profile is characterized by fluctuating peaks and troughs that can be associated with adverse effects and suboptimal efficacy, respectively. Similar fluctuating peaks and troughs are evident in the release profile of certain once-daily, extended release (ER) methylphenidate formulations (e.g., Focalin XR, Aptensio XR). A product with a slower initial increase in MPH exposure followed by sustained MPH plasma concentrations at therapeutic levels for the majority of the waking day can allow for higher tolerable doses that may afford greater efficacy for treating IH

Furthermore, there remains a need in the art for forms of d-methylphenidate that can promote awakening in the morning prior to taking any new doses and prior to taking any other pharmaceuticals.

### BRIEF SUMMARY OF THE INVENTION

The present technology provides methods of treating sleep disorders in a patient suffering therefrom, wherein the methods comprise administering to that patient a nightly dose of a therapeutically effective amount of a particular conjugate of d-methylphendiate without the need for a second-line treatment. The present technology additionally provides methods of treating sleep disorders in a patient suffering therefrom, wherein the methods comprise administering to that patient a nightly dose and a morning dose of a therapeutically effective amount of a particular conjugate of d-methylphendiate without the need for a second-line treatment.

In certain aspects, the present technology relates to a method of treating sleep disorders in a patient, the method comprising: administering to the patient before bedtime a composition comprising a therapeutically effective amount of a conjugate of d-methylphenidate having a structure of Formula I:

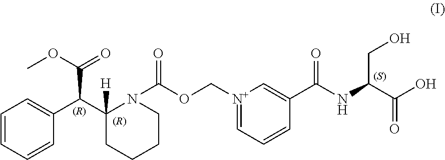

or a pharmaceutically acceptable salt thereof, wherein the composition comprising the conjugate of d-methylphenidate is administered about 0-3 hours before the bedtime of the patient.

In certain aspects, the present technology relates to a method of treating Idiopathic Hypersomnia in a patient, the method comprising: administering to the patient before bedtime a composition comprising a therapeutically effective amount of a conjugate of d-methylphenidate having a structure of Formula I:

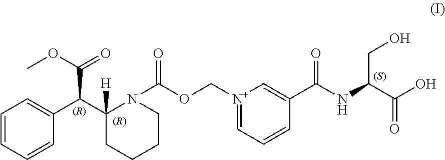

or a pharmaceutically acceptable salt thereof, wherein the composition comprising the conjugate of d-methylphenidate is administered about 0-3 hours before the bedtime of the patient.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the pharmaceutically acceptable salt is selected from acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further aspect, the conjugate of d-methylphenidate is serdexmethylphenidate chloride.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day. In a still further aspect, the therapeutically effective amount of the conjugate of d-methylphenidate is 240 mg per day.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further aspect. the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the Idiopathic Hypersomnia is associated with at least one additional sleep disorder selected from the group consisting of Excessive Daytime Sleepiness, Sleep Inertia, and Brain Fog.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the decrease in at least one sleep disorder test score is an at least 10% decrease in the Idiopathic Hypersomnia Severity Scale score. Alternatively, in certain aspects, the decrease is at least about 5%, about at least 10%, about 20%, about at least 30%, about at least 40%, and/or at least about 50%.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the at least 10% decrease in the Idiopathic Hypersomnia Severity scale is an at least 4 point decrease in the Idiopathic Hypersomnia Scale score. In a still further aspect, the at least 10% decrease in the Idiopathic Hypersomnia Severity Scale score is an at least 10 point decrease in the Idiopathic Hypersomnia Severity Scale score.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the 10% decrease results in lowering the level of Idiopathic Hypersomnia severity.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the patient in need thereof has a Idiopathic Hypersomnia Severity scale score of greater than 22 prior to starting the treatment.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the decrease in at least one sleep disorder test score is an at least 10% decrease in the Epworth Sleepiness Scale score.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the at least 10% decrease in the Epworth Sleepiness Scale score is an at least 2-point decrease in the Epworth Sleepiness Scale score. In a still further aspect, the at least 10% decrease in the Epworth Sleepiness Scale score is an at least 6 point decrease in the Epworth Sleepiness Scale score.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the patient in need thereof has an Epworth Sleepiness Scale score of greater than 10 prior to starting the treatment.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further aspect, the present technology relates to a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the patient reports an improved score on at least one sleep survey selected from the group consisting of Patient Global Impression of Severity, Clinical Global Impression of Severity, PROMISE29, Total Sleep Time, Total Nap Time, Psychomotor Vigilance Task (PVT), and Work Productivity and Activity Impairment Questionnaire: Specific Health Problem (WPAI:SHP).

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

In one aspect, the present technology relates to a method of treating Excessive Daytime Sleepiness in a patient, the method comprising: administering to the patient before bedtime a composition comprising a therapeutically effective amount of a conjugate of d-methylphenidate having a structure of Formula I:

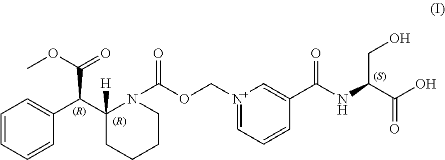

or a pharmaceutically acceptable salt thereof, wherein composition comprising a conjugate of d-methylphenidate is administered 0-3 hours before the bedtime of the patient.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the pharmaceutically acceptable salt is selected acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further aspect, the conjugate of d-methylphenidate is serdexmethylphenidate chloride.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day. In certain aspects, the therapeutically effective amount of the conjugate of d-methylphenidate is 240 mg per day.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day.

In a further aspect, the present technology relates to the method of treating Excessive Sleep Disorder in a patient, the method comprising administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further aspect. the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the Excessive Daytime Sleepiness is associated with at least one additional sleep disorder selected from the group consisting of Idiopathic Hypersomnia, Sleep Inertia, and Brain Fog.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the decrease in at least one sleep disorder test score is an at least 10% decrease in the Epworth Sleepiness Scale score. In a still further aspect, the decrease in at least one sleep disorder test score is an at least 10% decrease in the Epworth Sleepiness Scale score. Alternatively, in certain aspects, the decrease is at least about 5%, about at least 10%, about 20%, about at least 30%, about at least 40%, and/or at least about 50%.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the at least 10% decrease in the Epworth Sleepiness Scale score is an at least 2-point decrease in the Epworth Sleepiness Scale score. In a still further aspect, the at least 10% decrease in the Epworth Sleepiness Scale score is an at least 6 point decrease in the Epworth Sleepiness Scale score.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the patient in need thereof has an Epworth Sleepiness Scale score of greater than 10 prior to starting the treatment.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip.

In a further aspect, the present technology relates to the method of treating Excessive Daytime Sleepiness in a patient, the method comprising administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a further aspect, the present technology relates to the method of treating Idiopathic Hypersomnia in a patient, the method comprising administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

In a certain aspect, the present technology relates to a method of treating Sleep Inertia in a patient, the method comprising: administering to the patient in need thereof before bedtime a composition comprising a therapeutically effective amount of a conjugate of d-methylphenidate having a structure of Formula I:

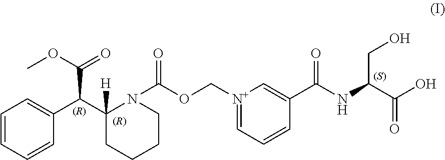

or a pharmaceutically acceptable salt thereof, wherein composition comprising a conjugate of d-methylphenidate is administered 0-3 hours before the bedtime of the patient in need thereof.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the pharmaceutically acceptable salt is selected from acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further aspect, wherein the conjugate of d-methylphenidate is serdexmethylphenidate chloride.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day. In certain aspects, the therapeutically effective amount of the conjugate of d-methylphenidate is 240 mg per day.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day. In certain aspects, the therapeutically effective amount of serdexmethylphenidate chloride is 257.0 mg per day.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further aspect. the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the Sleep Inertia is associated with at least one additional sleep disorder selected from the group consisting of Excessive Daytime Sleepiness, Idiopathic Hypersomnia, and Brain Fog.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS). In a still further aspect, the decrease in at least one sleep disorder test score is an at least 10% decrease in the Sleep Inertia Visual Analog Scale (SI-VAS)score. In a still further aspect, the decrease in at least one sleep disorder test score is an at least 10% decrease in the Sleep Inertia Visual Analog Scale (SI-VAS)score. Alternatively, in certain aspects, the decrease is at least about 5%, about at least 10%, about 20%, about at least 30%, about at least 40%, and/or at least about 50%.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the decrease in at least one sleep disorder test score is an at least 10% decrease in the Sleep Inertia Visual Analog Scale (SI-VAS)score. In a still further aspect, the decrease in the Sleep inertia Visual Analog Scale (SI-VAS) score is an at least 5-point decrease in the Sleep Inertia Visual Analog Scale (SI-VAS)score. In a still further aspect, the at least 10% decrease in the Sleep Inertia Visual Analog Scale (SI-VAS)score is an at least 15 point decrease in the Sleep Inertia Visual Analog Scale (SI-VAS)score.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the patient in need thereof has an Sleep Inertia Visual Analog Scale (SI-VAS) score of greater than 50 prior to starting the treatment.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a further aspect, the present technology relates to the method of treating Sleep Inertia in a patient, the method comprising administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

In certain aspects, the present technology relates to a method of treating Brain Fog in a patient, the method comprising: administering to the patient before bedtime a composition comprising a therapeutically effective amount of a conjugate of d-methylphenidate having a structure of Formula I:

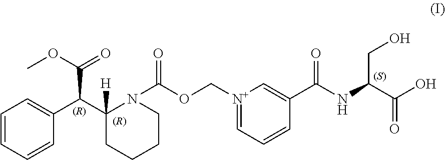

or a pharmaceutically acceptable salt thereof, wherein composition comprising a conjugate of d-methylphenidate is administered 0-3 hours before the bedtime of the patient.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the pharmaceutically acceptable salt is selected from acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further aspect, the conjugate of d-methylphenidate is serdexmethylphenidate chloride.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day In certain aspects, the therapeutically effective amount of the conjugate of d-methylphenidate is 240 mg per day.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day. In certain aspects, the therapeutically effective amount of serdexmethylphenidate chloride is 257.0 mg per day.

In a further aspect, the present technology relates to the method of treating Brain Fog in a patient, the method comprising administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further aspect. the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the Brin Fog is associated with at least one additional sleep disorder selected from the group consisting of Excessive Daytime Sleepiness, Sleep Inertia, and Idiopathic Hypersomnia.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS). In a still further aspect, wherein the decrease in at least one sleep disorder test score is an at least 10% decrease in the Brain Fog Symptom Scale score. Alternatively, in certain aspects, the decrease is at least about 5%, about at least 10%, about 20%, about at least 30%, about at least 40%, and/or at least about 50%.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the at least 10% decrease in the Brain Fog Symptom scale score is an at least 5 point decrease in the Brain Fog Symptom Scale score. In a still further aspect, the at least 10% decrease in the Idiopathic Hypersomnia Severity Scale score is an at least 20 point decrease in the Brain Fog Symptom Scale score.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the 10% decrease results in lowering the level of Brain Fog severity.

In a still further aspect, the present technology relates to the method of treating Brain Fog in a patient, the method comprising administering to the patient a composition comprising a therapeutically effective amount of a conjugate of d-methylphenidate having a structure of Formula I, wherein the patient in need thereof has a Brain Fog Symptom Scale score of greater than 35 prior to starting the treatment.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a still further aspect, the present technology relates to the method of treating Brain Fog in a patient, the method comprising administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

### BRIEF SUMMARY OF THE DRAWINGS

FIG. 1 shows pharmacokinetic (PK) data for patients administered 240 mg of serdexmethylphenidate once daily either in the morning or evening.

FIG. 2 shows mean plasma concentrations of d-methylphenidate and area under the curve (AUC) data for patients administered 240 mg of serdexmethylphenidate once daily either in the morning or in the evening.

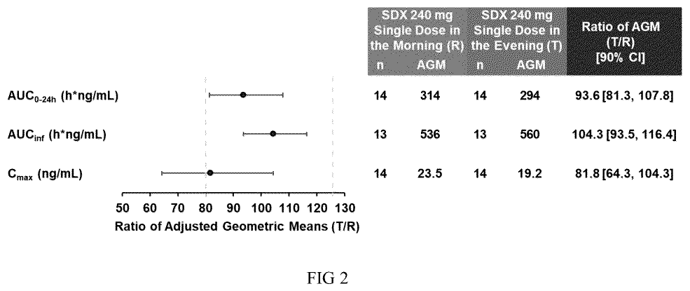

FIG. 3 shows the changes in mean Epworth Sleepiness Scale (ESS) scores during a seven week study comprising a 5-week titration period and a 2-week withdrawal period compared to placebo.

FIG. 4 shows the changes in mean Idiopathic Hypersomnia Severity Scale (IHSS) scores during a seven week study comprising a 5-week titration period and a 2-week withdrawal period compared to placebo.

FIG. 5 shows the changes in mean Sleep Inertia Visual Analog Scale (SI-VAS)) scores during a 7-week study comprising a 5-week titration period and a 2-week withdrawal period compared to placebo.

FIG. 6 shows the changes in mean Brain Fog Symptom Scale (BFS) scores during a 7-week study comprising a 5-week titration period and a 2-week withdrawal period compared to placebo.

FIG. 7 shows the total change in mean Epworth Sleepiness Scale (ESS) scores during the 2-week withdrawal period compared to placebo

FIG. 8 shows the changes in mean ESS scores from baseline during the 2-week withdrawal period

FIG. 9 shows the total change in mean Idiopathic Hypersomnia Severity Scale (IHSS) scores during the 2-week withdrawal period compared to placebo.

FIG. 10 shows the change in mean IHSS scores from baseline during the 2-week withdrawal period compared to placebo.

FIG. 11 shows the total change in mean SI-VAS scores during the 2-week withdrawal period compared to placebo.

FIG. 12 shows the change in mean SI-VAS scores from baseline during the 2-week withdrawal period compared to placebo.

FIG. 13 shows the total change in mean BFS scores during the 2-week withdrawal period compared to placebo.

FIG. 14 shows the change in mean BFS scores from baseline during the 2-week withdrawal period compared to placebo.

FIG. 15 shows the plasma concentration of d-methylphenidate following administration of 80 and 200 mg serdexmethylphenidate compared to Ritalin administered either as 2 40 mg doses 5 hours apart or a single 80 mg dose.

FIG. 16 shows d-methylphenidate plasma concentrations for serdexmethylphenidate administered once daily in the evening compared to twice daily.

FIG. 17 shows plasma concentrations and standard deviations of d-methylphenidate administered once daily in the evening (B) compared to once daily in the morning (A).

FIG. 18 shows plasma serdexmethylphenidate concentrations and standard deviations for serdexmethylphenidate administered once daily in the evening (B) compared to once daily in the morning (A).

FIG. 19 summarizes the Adverse Events (AEs) reported by study participants.

FIG. 20 show PK data for d-MPH plasma levels over a 24-hour window for both QD and BIG dosing schedules.

### DETAILED DESCRIPTION

In certain embodiments, the present technology relates to a method of treating a sleep disorder in a patient in need thereof, the method comprising administering to the patient before bedtime a composition comprising a conjugate of d-methylphenidate having the structure of Formula I:

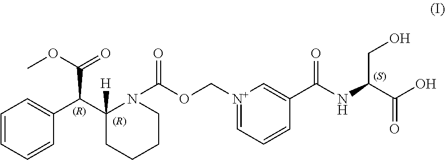

or a pharmaceutically acceptable salt thereof, wherein the pharmaceutical composition comprising a conjugate of d-methylphenidate is administered before the bedtime of the patient.

### INTRODUCTION

Conventionally, sleep disorders like IH are treated by administering several classes of drugs, including depressants, such as sodium oxybates, to induce sleep. However, such drugs can make it difficult for a patient with IH associated with Excessive Daytime Sleepiness (EDS) and/or Sleep Inertia (SI) to reach a full state of wakefulness following sleep. Additionally, these drugs have complicated dosing regimens and carry a risk developing drug dependence.

Stimulants, including methylphenidate (“MPH”), are believed to enhance the activity of the sympathetic nervous system and/or central nervous system (CNS). Stimulants such as MPH and the various forms and derivatives thereof are used for the treatment of a range of conditions and disorders predominantly encompassing, for example, sleep disorders, including, but not limited to narcolepsy, excessive daytime sleepiness, central hypersomnolence disorders and/or wakefulness. Methylphenidate is a psychostimulant which is a chain substituted amphetamine derivative. Similar to amphetamine and cocaine, methylphenidate targets the central nervous system, specifically the dopamine transporter (DAT) and norepinephrine transporter (NET). Methylphenidate is thought to act by increasing the concentrations of dopamine and norepinephrine in the synaptic cleft by inhibiting their reuptake into the axon terminal at both the dopamine transporter (DAT) and the norepinephrine transporter (NET).

The presently described technology unexpectedly discovered that administering the conjugate of d-methylphenidate having a structure of Formula 1 before the patient goes to bed results in reducing the symptoms of IH, EDS, SI, and BF, upon waking in the morning without causing any or significant insomnia during the night. Conventionally, administering a stimulant before bedtime causes restlessness and insomnia resulting in excessive sleepiness during the waking hours of the following day.

### General Definitions

“A” and “an” as it relates to the present technology, means the singular form, but includes the plural form unless clear from the context.

“About” as it related to the present technology means, as it applied to measured quantities, +/−10% of the stated measured value; for example, “about 100 mg” means 100 mg+/−10%, i.e. 90-110 mg. Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “or” means, and is used interchangeably with, the term “and/or,” unless context clearly indicates otherwise.

As used herein, the term “such as” means, and is used interchangeably with, the phrase “such as, for example” or “such as but not limited.”

As used herein, the term “subject” means a human or animal, including but not limited to a human or animal patient.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

AUC₀₋₂₄ₕ, as it relates to the present technology, is a term used in pharmacokinetics to describe the area under the curve in a plot of drug concentration in blood, serum, or plasma vs time from time=0 (or pre-dose) to 24 hours after dosing.

AUC_{inf}, as it relates to the present technology, is a term used in pharmacokinetics to describe the area under the curve in a plot of drug concentration in blood, serum, or plasma vs time from time=0 (or predose) to infinity.

“BID” or “bis in die” as it relates to the present technology means a twice daily dosage regimen.

“Bioavailability,” as it relates to the present technology, means the proportion of a drug or other substance that enters the circulation over time when introduced into the body and so is able to have an active effect.

“Cₘₐₓ,” used hereinafter, is a term used in pharmacokinetics and refers to the maximum (or peak) plasma concentration that a drug achieves in a specified compartment or test area of the body after the drug has been administered and before the administration of a second dose.

“Daily dosage,” as it relates to the present technology, means the total amount of drug administered in a human or animal subject in a single day or 24-hour period.

As used herein, the phrases such as “decreased,” “reduced,” “diminished” or “lowered” are meant to include at least about a 10% change as it relates to, for example, pharmacological activity, area under the curve (AUC), and peak plasma concentration (Cₘₐₓ), Epworth Sleep Score (ESS), Idiopathic Hypersomnia Severity Scale (IHSS), Sleep Inertia Visual Analog Scale (SI-VAS), and/or Brain Fog Severity (BFS). For instance, the change may also be greater than about 10%, about 15%, about 20%, about 25%, about 35%, about 45%, about 55%, about 65%, about 75%, about 85%, about 95%, about 96%, about 97%, about 98%, about 99%, or increments therein.

The use of the term “dose” means the total amount of a drug or active component taken each time by an individual subject. Once that enters the systemic circulation over time when introduced into the body.

“Patient,” as it related to the present technology, means a human or animal subject in need of treatment.

“Pharmaceutical composition” or “pharmaceutical compositions,” as it relates to the present technology, means formulations for administration to the patient including solid or liquid oral formulations, parenteral formulations, suppositories, dermal patches, and topical ointments.

“Pharmaceutically effective amount” as used herein means an amount that has a pharmacological effect. A “pharmaceutically acceptable salt” as used herein is a salt of the d-methylphenidate conjugate or unconjugated methylphenidate or both which, when used in a pharmaceutically effective amount, has at least one pharmacological effect.

As used herein, the term “prodrug” refers to a substance that is inactive or has reduced pharmacological activity but is converted to an active drug by a chemical or biological reaction in the body. In the present technology, the prodrug is a conjugate of at least one drug, d-methylphenidate, a linker, and a nicotinoyl-L-serine moiety. Thus, the conjugates of the present technology are prodrugs and the prodrugs of the present technology are conjugates.

Prodrugs are often useful because, in some embodiments, they may be easier to administer or process than the parent drug. They may, for instance, be more bioavailable by oral administration whereas the parent drug is not. The prodrug may also have improved solubility in water and/or other solvents over the parent drug. A prodrug may also deliver the active parent drug and a more extended-release or delayed fashion. An embodiment of a prodrug would be a d-methylphenidate conjugate that is metabolized to the active moiety. In certain embodiments, upon in vivo administration, a prodrug is chemically or biologically hydrolyzed to release the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug is designed to alter the metabolism or the transport characteristics or the pharmacokinetics of a drug—the changes typically varying with route of administration—in certain embodiments, to mask side-effects or toxicity, to improve bioavailability and/or water solubility, to improve the flavor of a drug or to alter other characteristics or properties of a drug in other discrete embodiments.

“QD” or “quaque die” as it relates to the present technology means a once a day dosage regime.

As used herein, the term “subject” means a human or animal, including but not limited to a human or animal patient.

As known to those skilled in the art, the term “Steady State” means the state in which the overall intake of a drug is in approximate dynamic equilibrium with its elimination. At steady state, total drug exposure does not change significantly between successive dosing periods. Steady state is typically achieved following a time period about 4-5 times the half-life of a drug after regular dosing was started.

“Therapeutically effective amount” as used herein means an amount effective for treating a disease or condition or the underlying symptoms thereof.

A “therapeutically acceptable salt” as used herein is a pharmaceutically acceptable salt of the d-methylphenidate conjugate or unconjugated methylphenidate or both in the composition of the present technology, which, when used in a therapeutically effective amount, is effective for treating a disease, condition, or syndrome.

Tₘₐₓ, used hereinafter, is the term used in pharmacokinetics to describe the time at which the Cₘₐₓ is observed.

“Treating” as it relates to the present technology means any of the following: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Unit dose form” as it relates to the present technology means a single entity of a solid therapeutic dosage form (e.g., 1 capsule, 1 tablet) or a single volume dispensed from a non-solid dosage form (e.g., 5 mL of a liquid or syrup). Such a unit dose form can be from about 0.1 mg to about 500 mg per day, alternatively from about 0.1 mg to about 400 mg per day, about 0.1 mg to about 300 mg per day, alternatively about 1 mg to about 100 mg per day, alternatively about 5 mg to about 80 mg per day, alternatively about 10 mg to about 40 mg per day, alternatively about 10 mg to 200 mg per day, alternatively about 20 mg to about 120 mg per day, alternatively about 30 mg to about 100 mg per day, alternatively about 40 mg to about 80 mg per day, alternatively about 50 mg to about 70 mg per day, alternatively about 20 mg to about 40 mg per day, alternatively about 20 mg to about 60 mg per day, alternatively about 10 mg to about 50 mg per day, alternatively about 20 mg per day, alternatively about 40 mg per day, alternatively about 60 mg per day, alternatively 80 mg per day, alternatively 100 mg per day, alternatively 160 mg per day, alternatively 240 mg per day, alternatively 320 mg per day.

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

Serdexmethylphenidate chloride is summarized in the following table:

| | |
|---|---|
| Active Pharmaceutical | Serdexmethylphenidate (SDX) Chloride, a prodrug |
| Ingredient | of d-threo-methylphenidate (d-MPH) |
| Chemical Name: | 3-[[[(1S)-1-carboxy-2- |
| | hydroxyethyl]amino]carbonyl]-1-[[[[(2R)-2-[(1R)- |
| | 2-methoxy-2-oxo-1-phenylethyl]-1- |
| | piperidinyl]carbonyl]oxy]methyl]pyridinium |
| | chloride |
| Molecular Weight: | 535.98 g/mol (chloride salt) |
| | 500.53 g/mol (base) |
| Chemical Structure: | 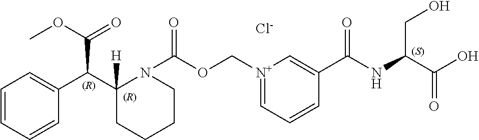 |
| | SDX Drug Substance |
| | C₂₅H₃₀ClN₃O₈ |
| | M.W.: 535.97 g/mol |
| Dosage Form, Route of | Solid oral dosage form at 80, 160, 240, and 320 mg |
| Administration, and Dosing | |
| Regimen | |

Serdexmethylpheni date as it relates to the present technology is a prodrug of d-methylphenidate. As a person of ordinary skill in the art will understand, drug products are considered pharmaceutical equivalents if they contain the same active ingredient(s), are of the same dosage form, route of administration, and are identical in strength or concentration. Pharmaceutically equivalent drug products are formulated to contain the same amount of active ingredient in the same dosage form and to meet the same or compendial or other applicable standards (i.e., strength, quality, purity, and identity), but they may differ in characteristics such as shape, scoring configuration, release mechanisms, packaging, excipients (including colors, flavors, preservatives), expiration time, and, with certain limits, labeling. Drug products are considered to be therapeutic equivalents only if they are pharmaceutical equivalents and if they can be expected to have the same clinical effect and safety profile when administered to patients under the conditions specified in the labeling.

The d-methylphenidate prodrug or conjugate of the present technology can be either a positively charged (cationic) molecule, or a pharmaceutically acceptable anionic or cationic salt form or salt mixtures with any ratio between positive and negative components. These anionic salt forms can include, but are not limited to, for example, acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. In the preferred embodiments, the anionic salt form is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite.

In some embodiments, the salt form of the conjugate is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, and nitrite. In some embodiments, the salt form of the unconjugated methylphenidate is selected from the group consisting of hydrochloride, hydrobromide, hydroiodide, formate, mesylate, tartrate, salicylate, sulfate, citrate, nitrate, hydrogen sulfite, propionate, benzene sulfonate, and acetate. The cationic salt forms can include, but are not limited to, for example, sodium, potassium, calcium, magnesium, lithium, cholinate, lysinium, or ammonium.

### Synthetic Scheme

A synthetic scheme for preparing d-MPH-CO₂CH₂-nicotinoyl-L-Ser is as follows:

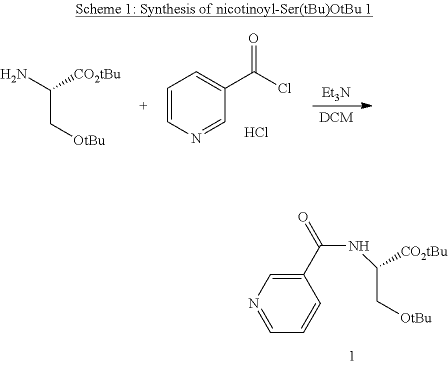

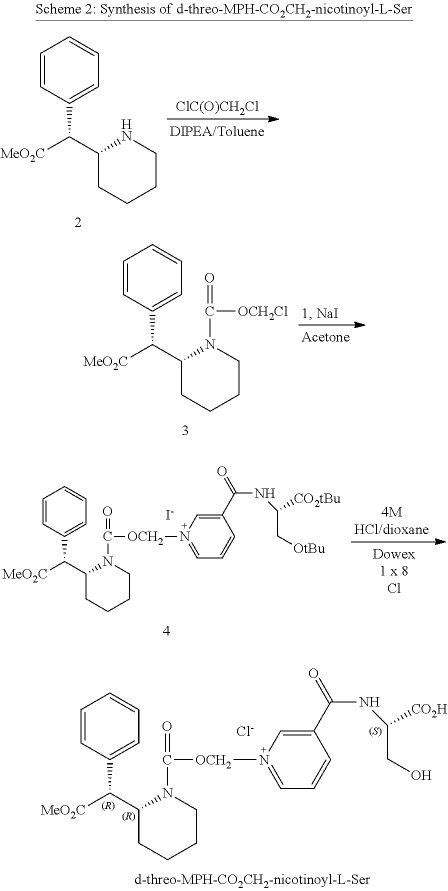

### 1) Nicotinoyl-Ser(tBu)OtBu 1:

To 0-tert-Butyl-L-Serine tert-butyl ester (H-Ser(tBu)OtBu, 5.305 g, 23.17 mmol) in DCM (250 mL) was added Et₃N (5.329 g, 53.29 mmol, 2.3 eq.). The flask was cooled in an ice-water bath (˜5° C.). Nicotinoyl chloride hydrochloride (4.331 g, 24.33 mmol, 1.05 eq.) was added in 7 portions over 1 hr. After adding, the water bath was removed and the reaction was stirred for another hour. 60 mL of 5% NH₄Cl was added to quench the reaction. The DCM layer was further washed with brine (60 mL) and dried over Na₂SO₄. The product was purified by column (hexanes:ethyl acetate, 1:1.3). 6.977 g of syrup was obtained. The yield was 93.4% and the purity was 98%.

b. 2) (2R, 2′R)-(+)-Methylphenidate hydrochloride 2 (d-threo isomer):

2 was made by resolution of d-threo-Methylphenidate hydrochloride 2 with O,O′-dibenzoyl-D-(+)-tartaric acid according to the method developed by Mahavir Prashad (Tetrahedron: Asymmetry 1999, 10, 3111). The yield was 40-42%.

c. 3) (R)-Chloromethyl-2-((R)-2-methoxy-2-oxo-1-phenylethyl)piperidine-1-carboxylate 3:

d-(+)-threo Methylphenidate hydrochloride (d-MPH·HCl) 2 (8.093 g, 30 mmol) in toluene (150 mL) was added DIPEA (12.4 mL, 75 mmol) under ice-water bath (˜5° C.). Then chloromethyl chloroformate (5.029 g, 39 mmol) in toluene (50 mL) was added over 20 min at about 5° C. After adding, the reaction was stirred at about 5-10° C. for 40 min. 5% NH₄Cl (50 mL) was added to quench the reaction. The toluene layer was separated, washed with brine (50 mL) and dried over Na₂SO₄. Solvent was evaporated to give crude 3, which was purified by silica gel chromatography column (hexanes:ethyl acetate, 3:1) to give 9.833 g of syrup (solidified when stored in freezer) and the yield was quantitative.

d. 4) 3-((S)-1-carboxy-2-hydroxyethylcarbamoyl)-1-(((R)-2-(2-(R)-methoxy-2-oxo-1-phenylethyl)piperidine-1-carbonyloxy)methyl)pyridium chloride d-threo-MPH-CO₂CH₂-nicotinoyl-L-Ser:

Nicotinoyl-Ser(tBu)OtBu 1 (0.322 g, 1 mmol) and carbamate 3 (0.355 g, 1.09 mmol, 1.09 eq.) were dissolved in acetone (10 mL). Then NaI (0.161 g, 1.08 mmol, 1.08 eq.) was added.

The reaction was refluxed for 1.5 hr. Upon cooling to room temperature, the reaction mixture was kept at room temperature for 2 hr. The solid (NaCl) was filtered off. The filtrate was concentrated and dried over vacuum for 1 hr to give amorphous solid 0.778 g. The solid in 4 M HCl/dioxane (5 mL) was stirred at room temperature for 2 hr.

Solvent was evaporated and the remaining was coevaporated with DCM (2 times 6 mL), and then dried over vacuum for 1 hr. to give amorphous solid 0.667 g. It was dissolved in 10 mL of ethanol and treated with resin twice (2 times 1 g, Dowex 1×8, 200-400, Cl form, prewashed with water and ethanol, wet). The filtrate after resin treatment was concentrated and dried over vacuum to give amorphous solid 0.617 g. The solid was dissolved in 10 mL of IPA with heating and then 5 mL of IPAc was added. Crystals formed gradually. After 3 hr., solid was collected and washed with IPA/IPAc (2:1, 3 times 1 mL), dried over vacuum. 437 mg of white solid (d-MPH-CO₂CH₂-nicotinoyl-L-Ser chloride) was obtained. The yield was 81.5% and the purity was 97.6%.

e. Preparation of cationic species of d-MPH-CO₂CH₂-nicotinoyl-L-Ser:

The chloride salt of d-MPH-CO₂CH₂-nicotinoyl-L-Ser is dissolved in water. The resulting solution contains free d-MPH-CO₂CH₂-nicotinoyl-L-Ser in cationic form.

In one embodiment, the present disclosure describes a composition comprising a therapeutically effective amount of a conjugate of d-methylphenidate having a structure of Formula I:

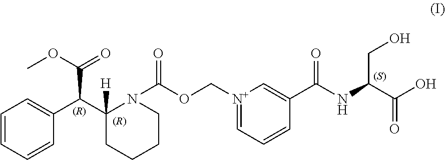

or a pharmaceutically acceptable salt thereof.

In one embodiment, the present disclosure describes a composition comprising a therapeutically effective amount of a conjugate of d-methylphenidate having a structure of Formula I, wherein the composition is formulated for oral administration, parenteral administrations, suppositories, and topical administration.

In one embodiment, the present disclosure describes a composition comprising a therapeutically effective amount of a conjugate of d-methylphenidate having a structure of Formula I, wherein the composition is an oral dosage formulation. In a still further embodiment, the oral dosage formulation is a solid oral dosage formulation. Alternatively, the composition is a liquid oral dosage.

Liquid dispersions for oral administration may be solutions, syrups, emulsions, or suspensions. The syrups may contain as carrier, for example, saccharose or saccharose with glycerol and/or mannitol and/or sorbitol. In particular a syrup for diabetic subjects can contain as carriers only products, for example sorbitol, which do not metabolize to glucose or which metabolize only a very small amount to glucose. The suspensions and the emulsions may contain a carrier, for example a natural gum, agar, sodium alginate, pectin, methylcellulose, carboxymethylcellulose or polyvinyl alcohol.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable, flavoring, preserving, suspending, thickening or emulsifying agents can be included.

Oral formulations of the present technology can also be included in a solution, a suspension or a slurry in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which is then placed in the feeding tube of a subject who is unable to swallow.

Solid dosage forms can optionally include one or more of the following types of excipients: antiadherents, binders, coatings, disintegrants, gel forming agents, fillers, flavors and colors, glidants, lubricants, preservatives, sorbents and sweeteners.

Other compounds which may be included by admixture are, for example, medically inert ingredients, e.g., solid and liquid diluents, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate, hydrogenated oils, sodium stearyl fumarate, and/or polyethylene glycols; gelling agents such as colloidal clays, polyethylene oxide, hydroxypropyl methyl cellulose, or carbomers; thickening agents such as gum tragacanth or sodium alginate, binding agents such as starches, arabic gums, gelatin, methylcellulose, carboxymethylcellulose or polyvinylpyrrolidone (povidone); disintegrating agents such as starch, alginic acid, alginates, crospovidone, or sodium starch glycolate; effervescing mixtures; dyestuff; sweeteners; wetting agents such as lecithin, polysorbates, poloxamer, sorbitan monoesters, glyceryl monooleates, or lauryl sulfates; and other therapeutically acceptable accessory ingredients, such as humectants, preservatives, buffers and antioxidants, which are known additives for such formulations.

It should be understood that in addition to the ingredients particularly mentioned above, the formulations of the present technology can include other suitable agents, such as flavoring agents, preservatives, and antioxidants, among others. Such antioxidants would be food acceptable and could include, for example, vitamin E, carotene, BHT or other antioxidants.

The compositions of the present technology can be formulated into dosage forms that include but are not limited to a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip. Preferred oral administration forms are capsule, tablet, solutions and OTF. Suitable dosing vehicles for solutions or suspensions of the present technology include, but are not limited to, water, phosphate buffered saline (PBS), 10% Tween in water, and 50% PEG-400 in water.

Pharmaceutical compositions of the present technology may be manufactured by known methods. As a non-limiting example, dry blending of active ingredients and excipients followed by wet granulation processes and subsequent milling followed by final compression and coating can be used to produce weight proportional tablets from the same granulation blend.

In one embodiment, the present disclosure describes a composition comprising a therapeutically effective amount of a conjugate of d-methylphenidate having a structure of Formula I, wherein the composition further comprises least one other active pharmaceutical ingredient. In certain aspects, the at least one additional pharmaceutical ingredient is selected from stimulants, depressants, analgesics, and sleep medications. In certain aspects, the other active pharmaceutical ingredient is selected from methylphenidate, aripiprazole, atomoxetine, baclofen, clonidine, desipramine, dihydrotetrabenazine, guanfacine, haloperidol, levetiracetam, mecamylamine, etoclopramide, olanzapine, ondansetron, pergolide, pimozide, pramipexole, risperidone, selegiline, sulpiride, tetrabenazine, topiramate, ziprasidone, or one or more of additional amphetamine, amitriptyline, or any combination thereof. In certain aspects, the sleep medication selected from zolpidem, ramelteson, temazepam, triazolam, benzodiazepine, doxepin, estazolam, trazodone, zaleplon, eszopiclone, suvorexant, flurazepam, lorazepam, chloral hydrate, a barbiturate, an antidepressant, or combinations thereof.

In certain aspects, the other active ingredient is a sleep aid selected from melatonin, diphenhydramine, doxylamine succinate, and valerian.

In an embodiment, the present disclosure described a method of treating Idiopathic Hypersomnia (IH), in a patient in need thereof, the method comprising administering to the patient before bedtime a pharmaceutical composition comprising a therapeutically effective amount of the of a conjugate of d-methylphenidate having the structure of Formula I:

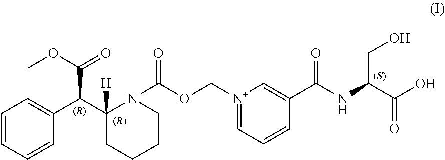

or a pharmaceutically acceptable salt thereof, wherein the pharmaceutical composition comprising a conjugate of d-methylphenidate is administered 0-3 hours before the bedtime of the patient.

“Idiopathic hypersomnia” or “IH,” as it related to the preset technology, means a neurological disorder which is characterized primarily by excessive sleep and excessive daytime sleepiness (EDS). Idiopathic hypersomnia was first described by Bedrich Roth in 1976, and it can be divided into two forms: polysymptomatic and monosymptomatic. The condition typically becomes evident in early adulthood and most patients diagnosed with IH will have had the disorder for many years prior to their diagnosis. As of August 2021, an FDA-approved medication exists for IH called Xywav, which is oral solution of calcium, magnesium, potassium, and sodium oxybate. Additionally, several other treatments are commonly prescribed off-label (primarily FDA-approved narcolepsy medications). Idiopathic hypersomnia may also be referred to as IH, IHS, or primary hypersomnia and belongs to a group of sleeping disorders known as central hypersomnias, central disorders of hypersomnolence, or hypersomnia of brain origin. Diagnostic and Statistical Manual of Mental Disorders, Fourth Edition (DSM-IV) defines idiopathic hypersomnia as EDS without narcolepsy or the associated features of other sleep disorders. It occurs in the absence of medical problems or sleep disruptions, such as sleep apnea, that can cause secondary hypersomnia. Individuals with IH share common symptoms including excessive daytime sleepiness, sleep inertia, brain fog, and long sleep periods. Excessive daytime sleepiness is characterized by persistent sleepiness throughout the day and often a general lack of energy, even during the day after apparently adequate or even prolonged nighttime sleep. People with EDS nap repeatedly throughout the day and have strong urges to sleep while driving, working, eating, or conversing with others. Sleep inertia (also known as sleep drunkenness) is characterized by having extreme difficulty waking up and feeling an uncontrollable desire to go back to sleep. Brain fog is characterized by clouding of consciousness characterized by inattention, thought process abnormalities, comprehension abnormalities, and language abnormalities. These symptoms may affect performance on perception, memory, learning, executive functions, language, constructive abilities, voluntary motor control, attention, and mental speed. Individuals with IH may complain of forgetfulness, confusion, or inability to think clearly; and excessive sleep (9 hours or more over a full 24-hour period), without feeling refreshed after waking. Daytime naps can be up to several hours and are also unrefreshing.

Idiopathic Hypersomnia is a chronic, debilitating central disorder of hypersomnolence. People with IH experience excessive daytime sleepiness (EDS) (AASM 2023). Key symptoms of EDS include sleepiness that persists throughout most of the day, daily inability to stay awake, need for scheduled naps, difficulties with attention and vigilance, and automatic behaviors (Lammers 2020). The previous definition of IH (AASM 2023) overlooked the disabling symptoms experienced by many people, including depressive symptoms, impaired vigilance, automatic behaviors, impaired executive functioning (e.g., cognitive and attentional difficulties, poor memory), increased need for sleep and severe sleep inertia (i.e., “sleep drunkenness”) (Lammers 2020), which is experienced by up to 79.0% of people with IH (Trotti 2020). For people with IH, long (>1 hour) daytime naps that are typically unrefreshing (Vernet 2010, Trotti 2020), impaired executive functioning, fatigue, decreased alertness, depressive symptoms, and automatic behaviors contribute to overall disease burden and poor quality of life (Vernet 2010, Trotti 2020, Schneider 2023, Stevens 2023). Although not formally defined (Rosenberg 2024) nor listed as a symptom in the updated American Academy of Sleep Medicine (AASM) International Classification of Sleep Disorders 3rd Edition—Text Revision (ICSD-3-TR; AASM 2023), brain fog is reported by 82.6% people with IH (Trotti 2020) and is characterized by problems with concentration and memory, inattention, confusion, difficulty understanding spoken and written language, reduced mental acuity, and mental fatigue (Debowska 2024).

“A conjugate of d-methylphenidate,” as it relates to the present technology, means d-methylphenidate conjugated to a nicotinoyl-L-serine moiety to form a prodrug. In certain aspects of the present technology, the conjugate of d-methylphenidate has the threo- or (R)(R)(S)-stereoisomer configuration, wherein the α-carbon and the β-carbon (part of the piperidine ring) next to the carboxyl group in the methylphenidate moiety have an (R)-configuration and the α-carbon next to the carboxyl group in the serine moiety has the (S)-

“Before bedtime” as it relates to the present technology means within about three hours, within about two hours, within about 90 minutes, within about 60 minutes, within about 45 minutes, within about 30 minutes, within about 20 minutes, within about 15 minutes, within about 10 minutes, within about 5 minutes or immediately before the patient begins the process of going to sleep.

In a further embodiment, the present disclosure describes a method of treating Idiopathic Hypersomnia (IH) in a patient in need thereof, the method comprising administering to the patient before bedtime a composition comprising a therapeutically effective amount of a conjugate of d-methylphenidate having a structure of Formula I, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further embodiment, the conjugate of d-methylphenidate is serdexmethylphenidate chloride.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day. In a still further embodiment, the therapeutically effective amount of the conjugate of d-methylphenidate is 240 mg per day.

In a further embodiment, the present disclosure discribes the method of treating Idiopathic Hypersomnia in a patient, the method comprising administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day. In a still further embodiment, the therapeutically effective amount of serdexmethylphenidate chloride is 257.0 mg per day.

In a further embodiment, the present disclosure describes the method of treating Idiopathic Hypersomnia in a patient, the method comprising administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further embodiment, the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient.

In an embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the Idiopathic Hypersomnia is associated with at least one additional sleep disorder selected from the group consisting of Excessive Daytime Sleepiness, Sleep Inertia, and Brain Fog.

Excessive Daytime Sleepiness, as it relates to the present technology, means symptoms characterized by persistent sleepiness and often a general lack of energy, even during the day after apparently adequate or even prolonged nighttime sleep. EDS can be considered as a broad condition associated with several sleep disorders where increased sleep is a symptom, or as a symptom of another underlying disorder like narcolepsy, circadian rhythm sleep disorder, sleep apnea, or idiopathic hypersomnia.

Some persons with symptoms of EDS, including those with hypersomnias like narcolepsy and idiopathic hypersomnia, are compelled to nap repeatedly during the day; fighting off increasingly strong urges to sleep during inappropriate times such as while driving, while at work, during a meal, or in conversations. As the compulsion to sleep intensifies, the ability to complete tasks sharply diminishes, often mimicking the appearance of intoxication. During occasional unique and/or stimulating circumstances, a person with EDS can sometimes remain animated, awake and alert, for brief or extended periods of time. EDS can affect the ability to function in family, social, occupational, or other settings. A proper diagnosis of the underlying cause and ultimately treatment of symptoms and/or the underlying cause can help mitigate such complications. According to the National Sleep Foundation, around 20 percent of people experience excessive daytime sleepiness.

“Brain Fog,” as it relates to the present technology, means a group of symptoms generally characterized by not being able to think clearly or concentrate. Additional symptoms include, but are not limited to, feeling confused, feeling disorganized, difficulty focusing, difficulty articulating thoughts, and lack of mental clarity. Without being bound to one particularly theory, brain fog can be caused, for example, by fatigue, lack of sleep, stress, and certain disorders including, for example, idiopathic hypersomnia, narcolepsy, attention deficit hyperactivity disorder (ADHD), COVID-19 (an infectious disease caused by the SARS-CoV-2 virus), multiple sclerosis, Parkinson's disease, and dementia.

“Sleep Inertia,” as it relates to the present technology, is a physiological state of impaired cognitive and sensory-motor performance that is present immediately after awakening. It persists during the transition of sleep to wakefulness, where an individual will experience feelings of drowsiness, disorientation, and a decline in motor dexterity. Impairment from sleep inertia may take several hours to dissipate. In the majority of cases, morning sleep inertia is experienced for 15 to 30 minutes after waking. Symptoms include, but are not limited to “grogginess”, as defined by a drowsy or disoriented state in which there is a dampening of sensory acuity and mental processing; impaired motor dexterity and decrease in cognitive ability. These gross impairments may be responsible for the associated increase in reaction time and drop in attentiveness; deficits in spatial memory; reports of heightened subjective fatigue; and reduced vigilance and a desire to go back to sleep.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

“Idiopathic Hypersomnia Severity Scale” or “IHSS,” as it relates to the present technology, means a self-administered 14 question survey. Respondents are asked to rate on a 4-point scale (0-3) the severity of IH symptoms such as prolonged and/or unrefreshing daytime and nighttime sleep; impaired daytime alertness, and sleep inertia. The total possible score is 50 if all questions are rated at the maximum score. IHSS scores are generally interpreted as follows: 0-12, mild; 13-25, moderate; 26-38, severe; and 39-50, very severe.

“Epworth Sleepiness Scale” or “ESS,” as it relates to the present technology, means a self-administered questionnaire with 8 questions. Respondents are asked to rate, on a 4-point scale (0-3), their usual chances of dozing off or falling asleep while engaged in eight different activities. Most people engage in those activities at least occasionally, although not necessarily every day. The ESS score (the sum of 8 item scores, 0-3) can range from 0 to 24. The higher the ESS score, the higher that person's average sleep propensity in daily life (ASP), or their ‘daytime sleepiness’. ESS score are generally interpreted as follows: 0-5, lower normal daytime sleepiness; 6-10 higher normal daytime sleepiness; 11-12, mild excessive daytime sleepiness; 13-15, moderate excessive daytime sleepiness; and 16-24, severe excessive daytime sleepiness. [https://epworthsleepinessscale.com/about-theess/]herein incorporated by reference.

“Sleep Inertia Visual Analog Scale” or “SI-VAS,” as it relates to the present technology, is a psychometric response scale to assess how difficult it was for the patient to wake up in the morning. The SI-VAS is a unipolar scale anchored at 0 (“very easy”) and 100 (“very difficult”).

“Brain Fog Symptom Score” or “BFS” as it relates to the present technology, means the total score collected with a 19-item questionnaire asking the respondents to describe their mental state while experiencing “Brain Fog” during the last week. The currently recognized 19 mental states of BF include forgetful, difficulty thinking, difficulty focusing, cloudy, difficulty finding the right words/communicating, mental fatigue, slow, mind went blank, spacey, difficulty processing what others say, exhausted, easily distracted, difficulty processing words read, confused, annoyed, sleepy, lost, detached, thoughts moving too quickly. Each item is answered based on a 0 to 4-point Likert scale where 0 means “strongly disagree” and 4 means “strongly agree”.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the decrease in at least one sleep disorder test score is an at least 10% decrease in the Idiopathic Hypersomnia Severity Scale score. Alternatively, in certain aspects, the decrease is at least about 5%, about at least 10%, about 20%, about at least 30%, about at least 40%, and/or at least about 50%.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the at least 10% decrease in the Idiopathic Hypersomnia Severity scale is an at least 4 point decrease in the Idiopathic Hypersomnia Scale score. In a still further aspect, the at least 10% decrease in the Idiopathic Hypersomnia Severity Scale score is an at least 10 point decrease in the Idiopathic Hypersomnia Severity Scale score. In certain embodiments, the at least 10% decrease in the Idiopathic Hypersomnia Severity Scale score is an at least 15 point decrease in the Idiopathic Hypersomnia Severity Scale Score.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the 10% decrease results in lowering the level of Idiopathic Hypersomnia severity. In certain embodiments, the at least 10% decrease is the Idiopathic Hypersomnia Severity Scale decreases the patient in need thereof's IH severity from “severe” to “moderate.” Alternatively, the at least 10% decrease is the Idiopathic Hypersomnia Severity Scale decreases the patient in need thereof's IH severity from “very severe” to “moderate.” the at least 10% decrease is the Idiopathic Hypersomnia Severity Scale decreases the patient in need thereof's IH severity from “severe” to “mild.” the at least 10% decrease is the Idiopathic Hypersomnia Severity Scale decreases the patient in need thereof's IH severity from “‘moderate” to “mild.”

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the patient in need thereof has a Idiopathic Hypersomnia Severity scale score of greater than 22 prior to starting the treatment. In certain embodiments, the patient in need thereof has a Idiopathic Hypersomnia Severity scale score of greater than 37 prior to starting the treatment.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the decrease in at least one sleep disorder test score is an at least 10% decrease in the Epworth Sleepiness Scale score.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the at least 10% decrease in the Epworth Sleepiness Scale score is an at least 2-point decrease in the Epworth Sleepiness Scale score. In a still further aspect, the at least 10% decrease in the Epworth Sleepiness Scale score is an at least 6 point decrease in the Epworth Sleepiness Scale score.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the patient in need thereof has an Epworth Sleepiness Scale score of greater than 10 prior to starting the treatment.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further embodiment, the present disclosure describes the method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the patient reports an improved score on at least one sleep survey selected from the group consisting of Patient Global Impression of Severity, Clinical Global Impression of Severity, PROMISE29, Total Sleep Time, Total Nap Time, Psychomotor Vigilance Task (PVT), and Work Productivity and Activity Impairment Questionnaire: Specific Health Problem (WPAI:SHP).

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a further embodiment, the present disclosure describes a method of treating Idiopathic Hypersomnia (IH), in a patient in need thereof, the method comprising administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

In a still further embodiment, the present disclosure described a method of treating Idiopathic Hypersomnia in a patient in need thereof, wherein the patient in need thereof reports an improved score on at least one questionnaire selected from the group consisting of Patient Global Impression of Severity, Clinical Global Impression of Severity, PROMISE-29, Total Sleep Time, Total Nap Time, Psychomoto Vigilance Task (PVT), and Work Productivity and Activity Impairment Questionaor: Specific Health Problem (WPAI:SHP).

“Patient Global Impression of Severity,” as it relates to the present technology, means a single-item questionnaire that measures the severity of insomnia symptoms. It's a patient-reported tool that can be used to assess insomnia severity over the previous week. The Patient Global Impression of Severity questionnaire asks the patient to rate their insomnia on a scale from 0 to 5, where 0 equates to “none” and 5 equates to “very severe.”

“Clinical Global Impression of Severity,” as it related to the present technology, means a questionnaire that clinicians use to rate a patient's sleep disorder severity. The Clinical Global Impression of Severity questionnaire is a single-item scale with seven response options that asks the clinician to rate the severity of the patient's symptoms on a scale of 1 to 7, where 1 equates to “normal” or “not ill,” and 7 equates to “among the most extremely ill patients.”

“PROMISE-29,” as it relates to the present technology, means a patient-reported outcome survey that assesses, among other things, sleep disturbance.

“Total Sleep Time,” as it relates to the present technology, means the total time spend sleeping during a 24-hour period.

“Total Nap Time,” as it relates to the present technology, means the total time spent napping during the day.

“Psychomotor Vigilance Task” or “PVT,” as it related to the present technology, means a test that measures how consistently someone responds to visual or auditory stimuli. It's a common tool for assessing sustained attention and alertness.

“Work Productivity and Activity Impairment Questionnaire: Specific Health Problem” or “WPAI:SHP,” as it relates to the present technology, means a 6-item instrument to measure impairments over the past 7 days in both paid work and unpaid work due to one's health. The patient responds to questions related to work and activity impairment due to health problems. The six questions measure absenteeism, presentism. And the impairments of unpaid activity due to health problems over the preceding 7 days.

In one embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I:

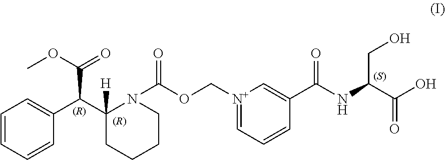

or a pharmaceutically acceptable salt thereof, wherein composition comprising a conjugate of d-methylphenidate is administered 0-3 hours before the bedtime of the patient.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the pharmaceutically acceptable salt is selected acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further aspect, the conjugate of d-methylphenidate is serdexmethylphenidate chloride.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day. In certain aspects, the therapeutically effective amount of the conjugate of d-methylphenidate is 240 mg per day.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day.

In a further embodiment, the present disclosure describes the method of treating Excessive Sleep Disorder in a patient, the method comprising: administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further embodiment, the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the Excessive Daytime Sleepiness is associated with at least one additional sleep disorder selected from the group consisting of Idiopathic Hypersomnia, Sleep Inertia, and Brain Fog.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the decrease in at least one sleep disorder test score is an at least 10% decrease in the Epworth Sleepiness Scale score. In a still further aspect, the decrease in at least one sleep disorder test score is an at least 10% decrease in the Epworth Sleepiness Scale score. Alternatively, in certain aspects, the decrease is at least about 5%, about at least 10%, about 20%, about at least 30%, about at least 40%, and/or at least about 50%.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the at least 10% decrease in the Epworth Sleepiness Scale score is an at least 2-point decrease in the Epworth Sleepiness Scale score. In a still further aspect, the at least 10% decrease in the Epworth Sleepiness Scale score is an at least 6 point decrease in the Epworth Sleepiness Scale score.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the patient in need thereof has an Epworth Sleepiness Scale score of greater than 10 prior to starting the treatment.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a further embodiment, the present disclosure describes a method of treating Excessive Daytime Sleepiness in a patient, the method comprising: administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

In one embodiment, the present disclosure describes a method of treating Sleep Inertia in a patient, the method comprising administering to the patient before bedtime a composition comprising a therapeutically effective amount of a conjugate of d-methylphenidate having a structure of Formula I:

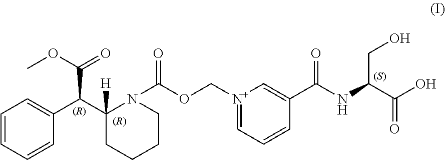

or a pharmaceutically acceptable salt thereof, wherein composition comprising a conjugate of d-methylphenidate is administered 0-3 hours before the bedtime of the patient.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the pharmaceutically acceptable salt is selected from acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further aspect, wherein the conjugate of d-methylphenidate is serdexmethylphenidate chloride.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day. In certain aspects, the therapeutically effective amount of the conjugate of d-methylphenidate is 240 mg per day.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day. In certain aspects, the therapeutically effective amount of serdexmethylphenidate chloride is 257.0 mg per day.

In a further embodiment, the present disclosure describes the method of treating Sleep Inertia in a patient, the method comprising: administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further embodiment, the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the Sleep Inertia is associated with at least one additional sleep disorder selected from the group consisting of Excessive Daytime Sleepiness, Idiopathic Hypersomnia, and Brain Fog.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS). In a still further aspect, the decrease in at least one sleep disorder test score is an at least 10% decrease in the Sleep Inertia Visual Analog Scale (SI-VAS)score. In a still further aspect, the decrease in at least one sleep disorder test score is an at least 10% decrease in the Sleep Inertia Visual Analog Scale (SI-VAS)score. Alternatively, in certain aspects, the decrease is at least about 5%, about at least 10%, about 20%, about at least 30%, about at least 40%, and/or at least about 50%.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the decrease in at least one sleep disorder test score is an at least 10% decrease in the Sleep Inertia Visual Analog Scale (SI-VAS)score. In a still further aspect, the decrease in the Sleep inertia Visual Analog Scale (SI-VAS) score is an at least 5-point decrease in the Sleep Inertia Visual Analog Scale (SI-VAS)score. In a still further aspect, the at least 10% decrease in the Sleep Inertia Visual Analog Scale (SI-VAS)score is an at least 15 point decrease in the Sleep Inertia Visual Analog Scale (SI-VAS)score.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the patient in need thereof has an Sleep Inertia Visual Analog Scale (SI-VAS) score of greater than 50 prior to starting the treatment.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a further embodiment, the present disclosure describes a method of treating Sleep Inertia in a patient, the method comprising: administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

In one embodiment, the present disclosure described a method of treating Brain Fog in a patient, the method comprising: administering to the patient before bedtime a composition comprising a therapeutically effective amount of a conjugate of d-methylphenidate having a structure of Formula I:

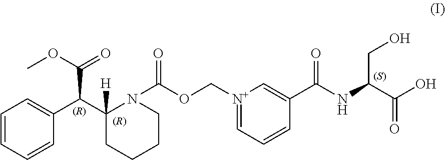

or a pharmaceutically acceptable salt thereof, wherein composition comprising a conjugate of d-methylphenidate is administered 0-3 hours before the bedtime of the patient.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the pharmaceutically acceptable salt is selected from acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate. In a still further aspect, the conjugate of d-methylphenidate is serdexmethylphenidate chloride.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the therapeutically effective amount of the conjugate of d-methylphenidate is between 0.1 mg and 500 mg per day. In a still further aspect, the therapeutically effective amount of the conjugate f d-methylphenidate is between 50 mg and 350 mg per day. In a still further aspect, the therapeutically effective amount of the conjugate f d-methylphenidate is selected from the group consisting of 80, 160, 240, or 320 mg per day. In certain aspects, the therapeutically effective amount of the conjugate of d-methylphenidate is 240 mg per day.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the therapeutically effective amount of a composition comprising serdexmethylphenidate chloride, wherein the therapeutically effective amount is selected from 87.5, 171.3, 257.0, or 342.7 mg per day. In certain aspects, the therapeutically effective amount of serdexmethylphenidate chloride is 257.0 mg per day.

In a further embodiment, the present disclosure describes the method of treating Brain Fog in a patient, the method comprising: administering to the patient before bedtime a composition comprising a therapeutically effective amount of a conjugate of d-methylphenidate having a structure of Formula I, wherein the composition comprising the conjugate of d-methylphenidate is administered once per day (QD) or twice per day (BID), wherein the twice per day administration is for each administration half the therapeutically effective amount. In a still further aspect, the composition comprising the conjugate of d-methylphenidate is administered once per day (QD). In yet a still further aspect, the composition comprising the conjugate of d-methylphenidate is administered once per day (QD) at night about 0-3 hours before bedtime of the patient. Alternatively, in yet a further embodiment, the composition comprising the conjugate of d-methylphenidate is administered once per day (QD) at night within about 0-2 hours, within about 0-90 minutes, within about 0-60 minutes, within about 0-45 minutes, within about 0-30 minutes, within about 0-20 minutes, within about 0-15 minutes, within about 0-10 minutes, within about 0-5 minutes or immediately before bedtime of the patient. In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the Brin Fog is associated with at least one additional sleep disorder selected from the group consisting of Excessive Daytime Sleepiness, Sleep Inertia, and Idiopathic Hypersomnia.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS). In a still further aspect, wherein the decrease in at least one sleep disorder test score is an at least 10% decrease in the Brain Fog Symptom Scale score. Alternatively, in certain aspects, the decrease is at least about 5%, about at least 10%, about 20%, about at least 30%, about at least 40%, and/or at least about 50%.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, the at least 10% decrease in the Brain Fog Symptom scale score is an at least 5 point decrease in the Brain Fog Symptom Scale score. In a still further aspect, the at least 10% decrease in the Idiopathic Hypersomnia Severity Scale score is an at least 20 point decrease in the Brain Fog Symptom Scale score.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the decrease is measured at least 1 week after an initial dosage. Alternatively, the patient score decrease is measured after 4 weeks, after 6 weeks, after 7 weeks, after 8 weeks, after 9 weeks, after 10 weeks, after 11 weeks, or after 12 weeks from the initial dose.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the 10% decrease results in lowering the level of Brain Fog severity.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the patient in need thereof has a Brain Fog Symptom Scale score of greater than 35 prior to starting the treatment.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the composition is an oral dosage formulation. In a still further aspect, the oral dosage formulation is a liquid dosage formulation selected from the group consisting of syrups, emulsions or suspensions. Alternatively, the oral dosage formulation is a solid dosage formulation selected from the group consisting of a sublingual, a gummy, a chewable tablet, a rapidly dissolving tablet, a tablet, a capsule (such as, including but not limited to, a soft-gel or liquid-filled capsule), a caplet, a troche, a lozenge, an oral powder, a thin strip, an oral thin film (OTF), and an oral strip

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

In a further embodiment, the present disclosure describes a method of treating Brain Fog in a patient, the method comprising: administering to the patient before bedtime a composition comprising a therapeutically effective amount of a conjugate of d-methylphenidate having a structure of Formula I, wherein the composition comprises at least one additional active pharmaceutical ingredient. In a still further aspect, the at least one additional active pharmaceutical ingredient is a sleep aid.

In some embodiments, the present technology provides pharmaceutical kits comprising a composition of the present technology that has increased water solubility than compared to the unconjugated d-methylphenidate. In some embodiments, the pharmaceutical kit comprises a specific amount of individual doses in a package, each dose comprising a pharmaceutically and/or therapeutically effective amount of the composition comprising the prodrug or conjugate of the present technology and unconjugated methylphenidate. The pharmaceutical kit may further include instructions for use. In some other embodiments, the kit comprises oral thin films or strips comprising the composition comprising the prodrugs or conjugates of the present technology and unconjugated methylphenidate. In some other embodiments, the kit comprises one or more blister packs containing the composition comprising the prodrug or conjugate of the present technology and unconjugated methylphenidate. It will be appreciated by one skilled in the art that, in some embodiments, the kit may include individual doses that have different dosage amounts.

The present technology provides pharmaceutical kits for the treatment or prevention of Excessive Daytime Sleepiness (EDS), Sleep Inertia (SI), and Brain Fog associated with Idiopathic Hypersomnia. The subject may be a patient in need thereof. As used herein the term animal is used in the veterinary sense and does not include humans. Suitable human subjects include neonatal subjects, pediatric subjects, adolescent subjects, adult subjects, geriatric subjects, elderly subjects and normative subjects. The kit comprises a specific amount of the individual doses in a package, each dose containing a pharmaceutically and/or therapeutically effective amount of at least one conjugate of d-methylphenidate of the present technology. The specified amount of individual doses may be from about 1 to about 100 individual dosages, alternatively from about 1 to about 60 individual dosages, alternatively from about 10 to about 30 individual dosages, including, about 1, about 2, about 5, about 10, about 15, about 20, about 25, about 30, about 35, about 40, about 45, about 50, about 55, about 60, about 70, about 80, about 100, and include any additional increments thereof, for example, about 1, about 2, about 5, about 10 and multiplied factors thereof, (e.g., about ×1, about ×2, about ×2.5, about ×5, about ×10, about ×100, etc.). One of skill in the art will appreciate that some embodiments of the kit of the present technology may include graduated individual doses (i.e. dose amounts that increase or decrease over a period of time), and/or a graduated dosing regimen, and instructions for use.

### EXAMPLES

The presently described technology and its advantages will be better understood by reference to the following examples. These examples are provided to describe specific embodiments of the present technology. By providing these specific examples, it is not intended to limit the scope and spirit of the present technology. It will be understood by those skilled in the art that the full scope of the presently described technology encompasses the subject matter defined by the claims appending this specification, and any alterations, modifications, or equivalents of those claims.

### Example 1—Pharmacokinetic Studies

Heathy adult subjects received single oral doses of 240 mg serdexmethylphenidate, either in the morning or at night, just before bedtime, in a randomized crossover fashion with a washout of 6 days between treatments. Subjects went to bed and nighttime conditions were maintained after administration of the evening dose. Multiple blood PK samples and safety parameters were collected after each administration. d-MPH was measured with a validated LC/MS-MS method. A total of 15 subjects (9 males/6 females) were randomized with 14 completing both treatments.

TABLE 1
Plasma d-MPH PK Parameters by Treatment

| | | | | | | |
|---|---|---|---|---|---|---|
| Treatment | Cₘₐₓ | Tₘₐₓ | T_{lag} | t_{1/2} | AUC_{0-24 h} | AUC_{inf} |
| Statistic | (ng/mL) | (h) | (h) | (h) | (h * ng/mL) | (h * ng/ml) |

SDX 240 mg single dose in the morning

| | | | | | | |
|---|---|---|---|---|---|---|
| N | 14 | 14 | 14 | 14 | 14 | 14 |
| Mean | 26.8 | 6.9 | 0.9 | 8.22 | 342 | 557 |
| (SD) | (13.9) | (1.7) | (0.5) | (3.07) | (139) | (266) |
| Median | | 6.5 | | | | |
| (min, max) | | (5.0, 12.0) | | | | |

SDX 240 mg single dose in the evening

| | | | | | | |
|---|---|---|---|---|---|---|
| N | 15 | 15 | 15 | 15 | 15 | 15 |
| Mean | 22.0 | 15.1 | 1.4 | 7.90 | 340 | 594 |
| (SD) | (9.44) | (5.3) | (0.4) | (2.49) | (145) | (223) |
| Median | | 16.0 | | | | |
| (min, max) | | (9.0, 24.0) | | | | |

SD = standard deviation

TABLE 2
Plasma PK Parameters of d-MPH after Single Oral Dose
Administrations of 240, 360, and 480 mg SDX Cl

| | 240 mg SDX Cl | | 360 mg SDX Cl | | 480 mg SDX Cl | |
|---|---|---|---|---|---|---|
| Parameters | N = 14 | | N = 10 | | N = 7 | |
| Tₘₐₓ (h) | 10.0 | (8.0-16.0) | 11.5 | (6.0-30.0) | 10.0 | (3.0-16.0) |
| Cₘₐₓ (ng/mL) | 18.2 | (50.6) | 23.9 | (42.6) | 43.0 | (26.9) |
| AUC_{0-24 h} (h*ng/mL) | 229 | (51.7) | 325 | (38.8) | 523 | (20.9) |
| AUCₗₐₛₜ (h*ng/mL) | 312 | (46.4) | 473 | (36.4) | 745 | (30.8) |
| AUC_{inf} (h*ng/mL) | 317 | (45.6) | 481 | (36.7)) | 760 | (31.4) |
| CL/F (L/h) | 384 | (39.5) | 387 | (55.7) | 297 | (30.0) |

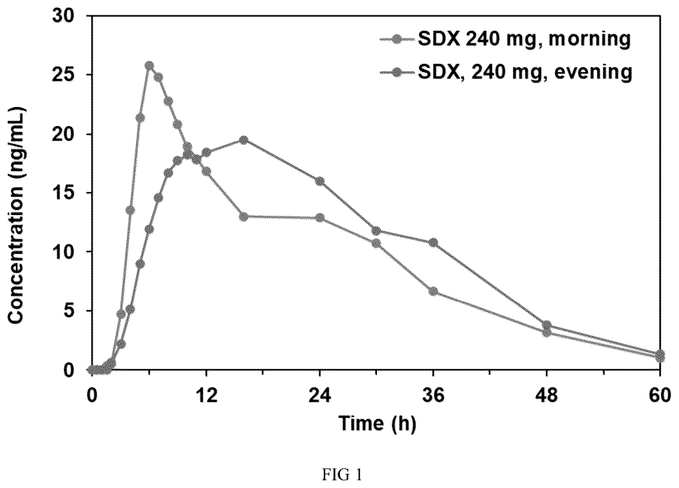

FIG. 1 shows the blood plasma concentration of d-methylphenidate measured by liquid chromatography-mass spectrometry (LC-MS/MS) for patients administered a single oral dose of 240 mg serdexmethylphenidate in the morning or just (shortly) before bedtime (evening trace). Pharmacokinetic data for these measurements are summarized in Table 1. Patients administered the morning dose showed a higher mean Cₘₐₓ (26.8 ng/mL) compared to patients administered the evening dose shortly before going to bed (22.0 ng/mL). Furthermore, mean time to peak d-methylphenidate concentration occurred after approximately 7 hours (Tₘₐₓ) in patients administered the morning dose compared to patients who received the dose shortly before going to bed (mean Tₘₐₓ of about 15 hours).

After the morning dose, d-MPH exposure was characterized by little or no d-MPH exposure for the first 4 hours followed by a gradual rise until about 7 hours post-dose (Tₘₐₓ) with a gradual decline thereafter. An even more gradual rise in d-MPH concentrations was seen after the nighttime dose resulting in a lower mean Cₘₐₓ and a statistically significantly longer median Tₘₐₓ of 16 hours compared to a median Tₘₐₓ of 6.5 hours after morning dosing.

The mean peak d-MPH plasma concentration was lower after the evening dose (22.0 ng/mL) compared to the morning dose (26.8 ng/mL) while total exposures (AUC_{inf}) were similar (594 and 557 h*ng/mL, respectively). Without wishing to be bound by theory, the large difference in Tₘₐₓ values for single daily dosages administered in the morning versus shortly before bed is likely due to a longer intestinal transit time and lower intestinal activity during the nighttime sleeping hours.

Table 2 shows the results of a Wilcoxon Signed Rant test used to compare the median Tₘₐₓ of d-methylphenidate between evening and morning dosing. The data demonstrated a statistically significant median difference in Tₘₐₓ of 9 hours for evening dosing vs morning dosing (p=0.0001).

TABLE 2
Wilcoxon Signed Rank Test of Plasma d-MPH Tmax

| | SDX 240 mg | | SDX 240 mg | | | | |
|---|---|---|---|---|---|---|---|
| | Single Dose in the | | Single Dose in the | | Median | 90% CI of | |
| Parameter | Morning (R) | | Evening (T) | | Difference | Median | |
| (Unit) | N | Median | N | Median | (T-R) | Differences | p-value |
| Tₘₐₓ (h) | 14 | 6.5 | 14 | 16.0 | 9.0 | 4.00, 10.00 | 0.0001 |

Note:
A Wilcoxon Signed Rank test was performed on the untransformed Tₘₐₓ. The exact p-value was calculated with a small sample size (N < 20). 90% CI was estimated using Hodges-Lehmann method. R = reference; T = test.

The unexpectedly slower metabolism of the conjugate of d-methylphenidate during sleep may provide a pharmacokinetic profile of the released d-methylphenidate that is uniquely suitable for evening dosing as the maximum rate of rise of d-methylphenidate concentrations coincides with the typical time of awakening after about 8 hours of sleep. At steady-state, administration of the same total daily dose once a day (QD) in the evening shortly before going to bed likely results in higher d-methylphenidate plasma concentrations at the time of awakening in the morning at about 6-12 hours after the last dose when compared to administration of half the total daily dose twice a day (BID) in the morning and in the evening. Once daily evening dosing before going to bed may therefore be more effective in reducing symptoms of sleep inertia associated with IH compared to BID dosing.

### Example 2—Sleep Study Investigations

A double-blind, placebo-controlled, randomized-withdrawal, dose-optimizing, multi-center study investigated the safety and efficacy of serdexmethylphenidate for the treatment of IH. Twenty-two (22) patients completed a 7-week study consisting of two parts. Part 1 of the trial comprised a five-week open-label titration phase during which patients were optimized to one of four total daily doses of serdexmethylphenidate (80, 160, 240, or 320 mg/day). About half the study participants were assigned to take the total daily dose of study drug once per day just before going to bed (QD dosing cohort). The other half was instructed to take half the daily dose of study drug in the morning after awakening and the second half of the daily dose just before going to bed (BID dosing cohort). The titration procedure during the 5-week open label period was conducted as follows: patient treatment was initiated with the lowest total daily dose (80 mg/day) regardless of dosing regimen. At subsequent clinical visits scheduled at intervals of about 1 week each, the dose of the patient was either decreased (except after the first week), maintained, or increased based on tolerability, clinical effect, and adverse events. Depending on the severities of any dose-limiting effects as determined by the clinician, the dose was decreased if possible or the patient was discontinued from the study. If the dose was tolerated by the patient and the clinician determined an opportunity to improve effectiveness, the dose of study drug was increased if possible. Otherwise, the dose was maintained in the subsequent week.

Part 2 of the trial comprised a two-week randomized (2:1), double-blind, withdrawal phase, during which two-thirds of the trial participants continued to receive their optimized dose while the remaining one-third received placebo. During the withdrawal phase, patients continued the same dosing regimen (QD or BID) they were assigned to at treatment initiation and adhered to during the titration phase.

The primary objective of the study was to evaluate the safety and tolerability of the conjugate of d-methylphenidate during treatment of adults with IH. The secondary objective was to explore the efficacy of the conjugate of d-methylphenidate in adults with IH.

Secondary efficacy endpoints included Excessive Daytime Sleepiness (EDS) assessed with the Epworth Sleepiness Scale (ESS). Additional secondary endpoints were based on the Idiopathic Hypersomnia Severity Scale (IHSS), daily rating of difficulty of waking up in the morning assessed with the Sleep Inertia Visual Analog Scale (SI-VAS), and rating of brain fog symptoms measured with an exploratory Brain Fog Scale (BFS). Scores on the ESS, IHSS, and BFS were based on a 1-week look-back period.

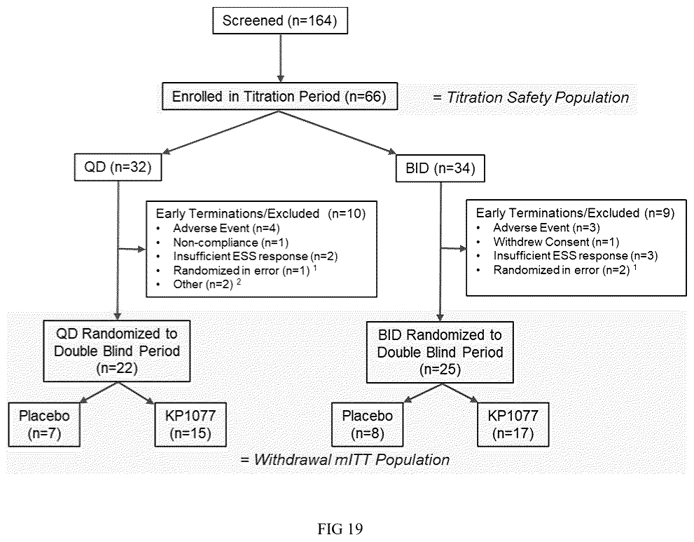

FIG. 19 shows the Phase 2 study design. Overall, 164 subjects were screened, with 66 subjects randomized in the Open-Label Titration Period: 32 subjects were randomized to receive the QD dosing regimen of SDX and 34 subjects were randomized to receive the BID dosing regimen of SDX (Table 7). A total of 57 subjects (86.4%) completed the Titration Period. Nine subjects (13.6%) discontinued the study during the Open-Label Titration Period: 6 subjects (9.1%) withdrew from the study due to an adverse event (AE), and 1 subject (1.5%) each withdrew consent, was non-compliant with study drug, or withdrew for other reasons (subject did not meet eligibility requirements). Of the 9 subjects (13.6%) overall who withdrew from the study during the Open-Label Titration Period, 5 subjects (15.6%) were in the SDX QD group, and 4 subjects (11.8%) were in the SDX BID group.

A total of 10 subjects (15.2%) were not eligible for the Double-Blind Withdrawal Period: The subjects who were randomized in error were excluded from the Withdrawal mITT Population. A total of 47 subjects (71.2%) were eligible for the withdrawal period: 22 subjects (68.8%) randomized to the SDX QD dosing regimen and 25 subjects (73.50%) randomized to the SDX BTD dosing regimen.

TABLE 3
Summary of Adverse Effects (AEs) reported by study participants.

| | | | |
|---|---|---|---|
| | SDX QD | SDX BID | Total |
| | (N = 32) | (N = 34) | (N = 66) |
| Preferred Term | n (%) | n (%) | n (%) |

| | | | | | |
|---|---|---|---|---|---|
| At least one TEAE | 19 | (59.4) | 21 | (61.8) | 40 (60.6) |
| Insomnia (all types) | 9 | (28.1) | 7 | (20.6) | 16 (24.2) |
| Headache | 4 | (12.5) | 2 | (5.9) | 6 (9.1) |
| Anxiety | 1 | (3.1) | 3 | (8.8) | 4 (6.1) |
| Decreased appetite | 3 | (9.4) | 1 | (2.9) | 4 (6.1) |
| nausea | 1 | (3.1) | 3 | (8.8) | 4 (6.1) |

TEAE = treatment-emergent adverse event

The results of the secondary efficacy endpoints are summarized in Table 3.

TABLE 3
Summary Results of ESS, IHSS, SI-VAS, and BFS

| | QD Cohort^{a} | | BID Cohort^{b} | |
|---|---|---|---|---|
| | Placebo | SDX | Placebo | SDX |
| Endpoint | (N = 7) | (N = 15) | (N = 8) | (N = 17) |

ESS, Mean (SD)

| | | | | | | | | |
|---|---|---|---|---|---|---|---|---|
| Titration Baseline | 18.3 | (3.50) | 16.9 | (2.36) | 16.5 | (3.30) | 16.5 | (2.72) |
| Randomization Baseline | 10.9 | (3.98) | 7.5 | (3.78) | 6.9 | (5.51) | 7.5 | (3.84) |
| (Week 5) | | | | | | | | |
| End of Withdrawal Period | 9.6 | (7.35) | 7.5 | (5.17) | 12.8 | (5.75) | 7.6 | (4.37) |
| (Week 7) | | | | | | | | |
| Change from Randomization | −1.3 | (3.95) | 0.0 | (4.17) | 5.9 | (4.26) | 0.1 | (4.87) |
| Baseline to Week 7 | | | | | | | | |

| | | |
|---|---|---|
| Difference in LS Mean (SE)^{c} | 0.5 (2.07) | −5.6 (1.88) |
| p-value | 0.818 | 0.005 |

IHSS, Mean (SD)

| | | | | | | | | |
|---|---|---|---|---|---|---|---|---|
| Titration Baseline | 33.4 | (10.26) | 35.4 | (4.22) | 36.6 | (5.78) | 34.3 | (6.42) |
| Randomization Baseline | 20.9 | (8.91) | 18.6 | (10.87) | 21.5 | (10.18) | 21.3 | (9.58) |
| (Week 5) | | | | | | | | |
| End of Withdrawal Period | 20.1 | (12.20) | 19.3 | (12.37) | 30.8 | (11.08) | 21.6 | (9.60) |
| (Week 7) | | | | | | | | |
| Change from Randomization | −0.7 | (8.69) | 0.7 | (6.86) | 9.3 | (11.65) | 0.3 | (7.61) |
| Baseline to Week 7 | | | | | | | | |

| | | |
|---|---|---|
| Difference in LS Mean (SE)^{c} | 0.9 (3.73) | −9.0 (3.48) |
| p-value | 0.208 | 0.104 |

SI-VAS, Mean (SD)

| | | | | | | | | |
|---|---|---|---|---|---|---|---|---|
| Titration Baseline | 76.0 | (15.08) | 68.8 | (17.29) | 75.8 | (10.11) | 68.5 | (18.82) |
| Randomization Baseline | 42.9 | (20.84) | 35.1 | (20.20) | 46.4 | (20.94) | 45.0 | (22.86) |
| (Week 5) | | | | | | | | |
| End of Withdrawal Period | 38.9 | (14.89) | 40.4 | (21.23) | 57.8 | (21.44) | 49.0 | (23.56) |
| (Week 7) | | | | | | | | |
| Change from Randomization | −4.0 | (21.08) | 5.9 | (15.13) | 11.4 | (13.69) | 1.1 | (7.35) |
| Baseline to Week 7 | | | | | | | | |

| | | |
|---|---|---|
| Difference in LS Mean (SE)^{c} | 8.2 (6.39) | −10.0 (5.98) |
| p-value | 0.208 | 0.104 |

Brain Fog, Mean (SD)

| | | | | | | | | |
|---|---|---|---|---|---|---|---|---|
| Titration Baseline | 47.9 | (12.64) | 52.3 | (16.88) | 55.6 | (10.90) | 52.4 | (17.44) |
| Randomization Baseline | 29.3 | (22.42) | 27.5 | (24.86) | 36.9 | (24.26) | 28.6 | (22.95) |
| (Week 5) | | | | | | | | |
| End of Withdrawal Period | 29.9 | (23.83) | 28.5 | (26.12) | 50.1 | (23.01) | 30.1 | (23.58) |
| (Week 7) | | | | | | | | |
| Change from Randomization | 0.6 | (9.80) | 0.9 | (20.79) | 13.3 | (12.66) | 1.5 | (17.34) |
| Baseline to Week 7 | | | | | | | | |

| | | |
|---|---|---|
| Difference in LS Mean (SE)^{c} | 0.0 (7.50) | −13.7 (7.07) |
| p-value | 0.995 | 0.060 |

SDX = serdexmethylphenidate
^{a}Patients received the total daily dose once per day in the evening shortly before going to bed.
^{b}Patients received half the total daily dose in the morning after awakening and the second half of the total daily dose in the evening shortly before going to bed.
^{c}ANCOVA with terms for randomization baseline score, treatment (active or placebo), regimen (BID or QD), and treatment by regimen as covariates

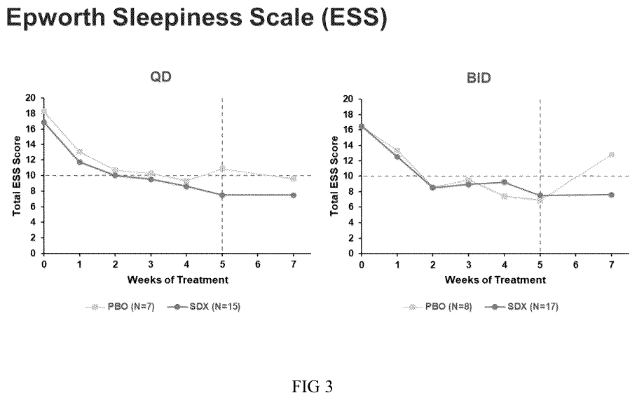

FIG. 3 shows the changes in Epworth Sleepiness Scale (ESS) for the QD and BID cohorts in patients treated with the conjugate of d-methylphenidate compared to placebo during the 5-week titration period and the 2-week withdrawal period. The minimally clinically important difference for the ESS scale is 2-3 points. The horizontal dashed line at an ESS score of 10 points marks the boundary between “normal” levels of daytime sleepiness and “excessive” levels of daytime sleepiness. Scores of 10 and less are generally considered “healthy” or “normal” levels of daytime sleepiness whereas scores greater than 10 are considered “excessive”. Both QD and BID cohorts showed significant decreases in mean ESS scores of about 8-10 points at the end of the 5-week titration period while being treated with the conjugate of d-methylphenidate. The mean ESS score did not meaningfully change (change of no more than about 0.1 points) in patients continuing treatment with the conjugate of d-methylphenidate during the 2-week withdrawal period.

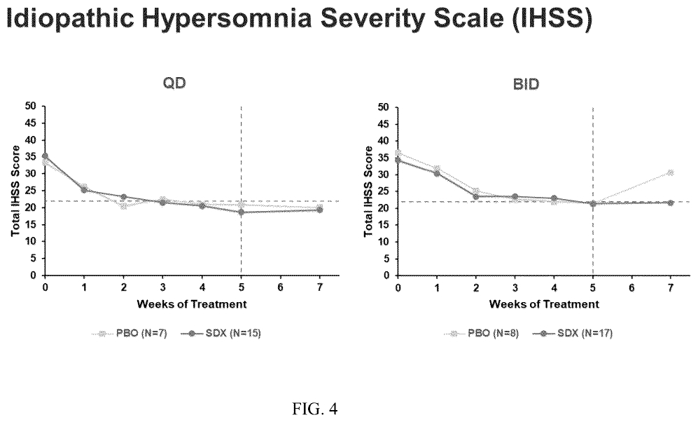

FIG. 4 shows the mean changes in Idiopathic Hypersomnia Severity Scale (IHSS)) for the QD and BID cohorts in patients treated with the conjugate of d-methylphenidate compared to placebo during the 5-week titration period and the 2-week withdrawal period. The minimally clinically important difference for the IHSS is 4 points. The horizontal dashed line at an IHSS score of 22 points marks the boundary between “normal” individuals and individuals that may have a sleep disorder with more severe symptoms of idiopathic hypersomnia. Scores of 22 and less are generally considered “healthy” or “normal” whereas scores greater than 22 indicate symptoms of idiopathic hypersomnia. During the 5-week titration period, treatment with the conjugate of d-methylphenidate resulted in a decrease in mean IHSS score of about 12-17 points in all patients. The mean IHSS score did not meaningfully change (change of no more than about 0.7 points) in patients continuing treatment with the conjugate of d-methylphenidate during the 2-week withdrawal period.

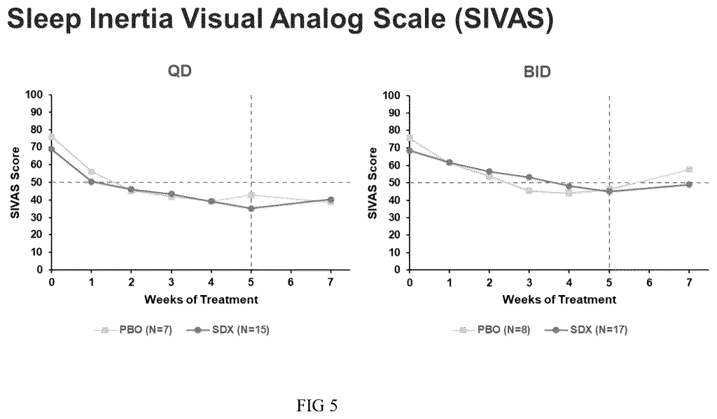

FIG. 5 shows the mean changes in Sleep Inertia Visual Analog Scale (SI-VAS) for the QD and BID cohorts in patients treated with the conjugate of d-methylphenidate compared to placebo during the 5-week titration period and the 2-week withdrawal period. The SI-VAS is a unipolar scale anchored at 0 (“very easy”) and 100 (“very difficult”). The horizontal dashed line at an SI-VAS score of 50 points represents the midpoint between individuals finding it very easy to awaken and individuals finding it very difficult to awaken in the morning. SI-VAS Patients on the QD dosing schedule reported a decrease of about 33 points in mean SI-VAS score over the 5-week titration period while being treated with conjugate of d-methylphenidate, while patients on the BID dosing schedule reported a decrease in mean SI-VAS of about 24-30 points. Patients of both QD and BID cohorts that continued treatment with the conjugate of d-methylphenidate showed small increases of about less than 6 points in mean SI-VAS score during the 2-week withdrawal period of about less than 5 points.

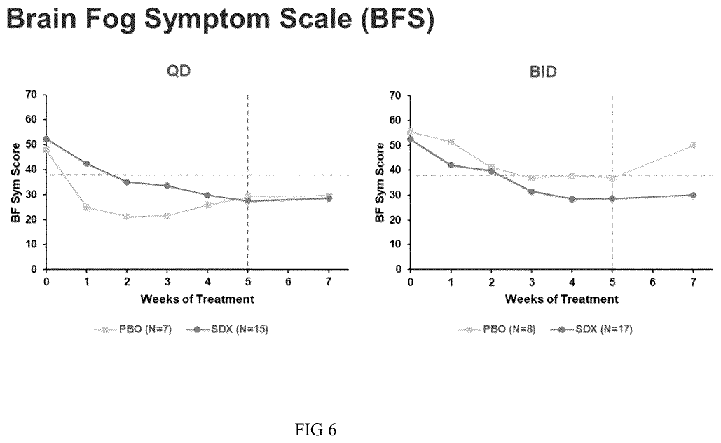

FIG. 6 shows the changes in mean Brain Fog Symptom (BFS) score for the QD and BID cohorts in patients treated with the conjugate of d-methylphenidate compared to placebo during the 5-week titration period and the 2-week withdrawal period. The BFS scale is based on a 19 item questionnaire where each question is scored on a 0-4 Likert scale where a score of 0 means “strongly disagree”, a score of 2 means “neutral”, and a score of 4 means “strongly agree”. Possible BFS total scores range from 0 to 76. The horizontal dashed line at a BFS score of 38 points represents the total BFS score of for “neutral” responses to all 19 questions. During the 5-week titration period, treatment with the conjugate of d-methylphenidate resulted in a decrease in mean BFS score of about 19-25 points in all patients. The mean BFS score did not meaningfully change (change of no more than about 1.5 points) in patients continuing treatment with the conjugate of d-methylphenidate during the 2-week withdrawal period.

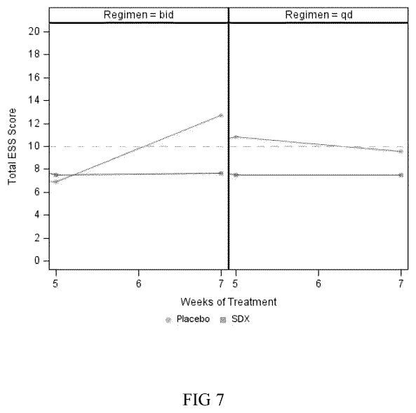

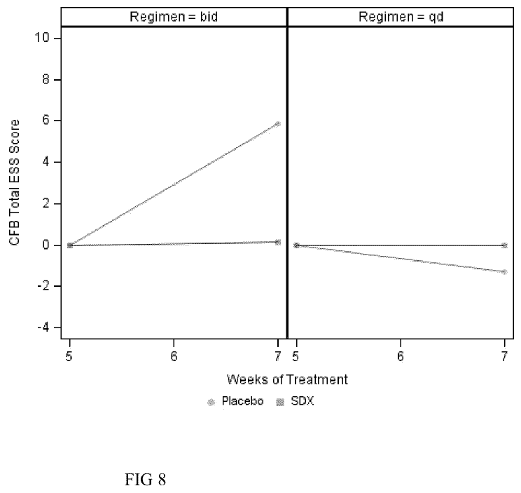

FIG. 7 and FIG. 8 show the overall mean change in ESS score and the mean change from baseline ESS score, respectively, during the 2-week withdrawal period. Patients who continued to receive the conjugate of d-methylphenidate showed about no change in ESS score over the withdrawal period.

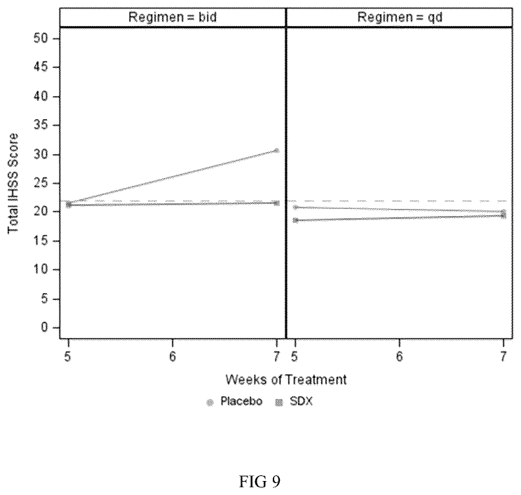

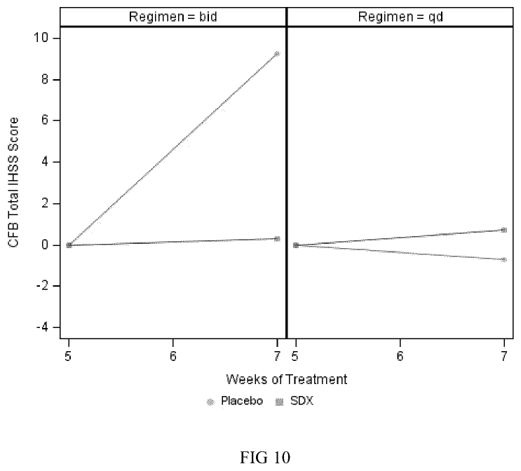

FIG. 9 and FIG. 10 shows the overall mean change in IHSS score and the mean change from baseline IHSS score, respectively, during the 2-week withdrawal period. Both QD and BID cohorts show little or no change in IHSS score during the 2-week withdrawal period.

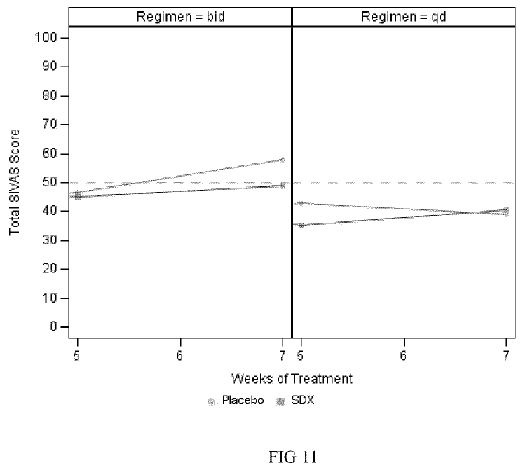

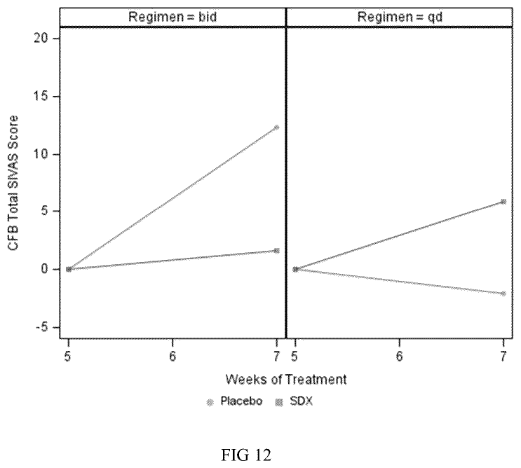

FIG. 11 and FIG. 12 show the overall mean change in SI-VAS score and the mean change from baseline SI-VAS score, respectively, during the 2-week withdrawal period. Both QD and BID cohorts showed a small increase in SI-VAS score of about less than 5 points during the 2-week withdrawal.

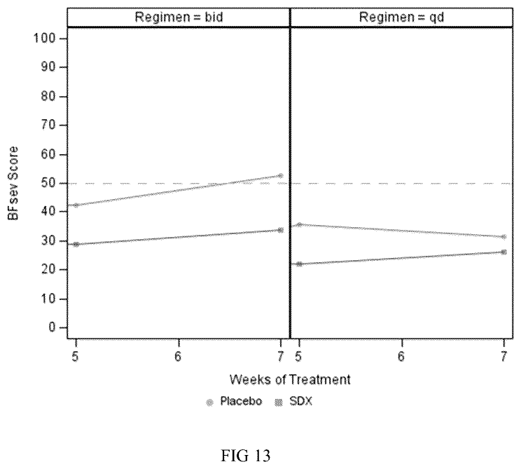

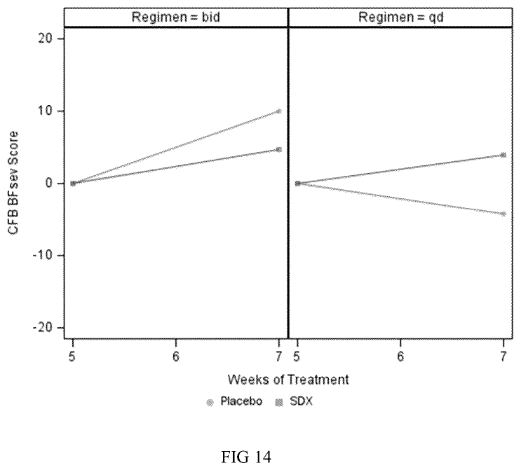

FIG. 13 and FIG. 14 show the overcall mean change in BFS score and the mean change from baseline BFS score, respectively, during the 2-week withdrawal period. Both QD and BID cohorts showed a small increase in BFS of about no more than 1.5 points score during the 2-week withdrawal period.

These results support treatment of EDS and SI in patients suffering from IH by administering a conjugate of d-methylphenidate. Both QD and BID patient cohorts showed clinically significant reductions in ESS, IHSS, SI-VAS, and BFS scores over the 5-week titration period. Furthermore, both QD and BID cohorts showed no or minimal score increases during the 2-week withdrawal period.

Unexpectedly, the release of d-methylphenidate following a single oral dose of the conjugate of d-methylphenidate in the evening shortly before going to bed was slower and the absorption phase was longer compared to oral administration of an equivalent dose of the conjugate of d-methylphenidate in the morning. Onset of first measurable concentration of d-methylphenidate in plasma was delayed by about 30 minutes in subjects dosed in the evening when compared to the morning. Time to maximal d-methylphenidate plasma concentrations (Tₘₐₓ) was about 8 hours longer in in subjects dosed in the evening when compared to the morning. Also unexpectedly, total plasma exposure to d-methylphenidate as measured by area under the curve (AUC) from time zero to 24 hours post-dose (AUC₀₋₂₄ₕ) and extrapolated AUC from time zero to infinity (AUC_{inf}) was similar for both evening and morning dosing of the conjugate of d-methylphenidate while the maximum d-methylphenidate plasma concentration (Cₘₐₓ) was about 20% lower following evening dosing compared to morning dosing of the conjugate of d-methylphenidate.

Not to be bound by one theory, it is assumed that the slowing of gastrointestinal (GI) motility during sleep after evening dosing prolongs the time for the conjugate of d-methylphenidate to reach the lower intestinal tract and colon where most of the oral dose is presumed to be metabolized to release d-methylphenidate. The longer transit time through the GI tract during the nighttime likely increases the drug distribution throughout the GI tract following the evening dose when compared to daytime administration of the conjugate of d-methylphenidate. As a result, the rate of release of d-methylphenidate from the conjugate of d-methylphenidate in the lower intestines is slower and plasma concentrations of d-methylphenidate increase more slowly after evening dosing compared to morning dosing. The longer absorption phase subsequently results in a lower d-methylphenidate Cₘₐₓ after evening dosing relative to morning dosing of the conjugate of d-methylphenidate. AUC of d-methylphenidate measured over at least 24 hours is comparable between evening and morning dosing as ultimately, a similar amount of the total oral dose passes through the GI tract regardless of dose timing.

### Example 3—Psychomotor Vigilance Test

Sleep Inertia was also measured by administering a Psychomotor Vigilance Test (PVT) which provides an objective functional measure that is less impacted by placebo effect as comparted to subjective measures of sleepiness. The PVT is a short and simple tool that is widely used to objectively measure behavioral alertness and sustained attention by recording reaction times (RT) to visual stimuli on an electronic device in sleep-deprived and sleep disorders.

### Example 4—Comparator Study with Ritalin

Ritalin is a racemate of d-methylphenidate approved for treating narcolepsy, which is a similar disorder of hypersomnolence characterized by significant excessive daytime sleepiness. The PK profiles of patient administered 80 mg or 200 mg of serdexmethylphenidate are compared to the PK profiles of patients administered 40 mg of Ritalin twice, 5 hours apart, and a single dose of 80 mg of Ritalin.

TABLE 5
Comparison of SDX pharmacokinetics to Ritalin.

| | | | | | Ritalin | | Ritalin | |
|---|---|---|---|---|---|---|---|---|
| | SDX 80 mg | | SDX 200 mg | | IR 80 mg | | LA 80 mg | |
| | (N = 14) | | (N = 14) | | (N = 14) | | (N = 14) | |
| Parameter | d-MPH | | d-MPH | | d-MPH | | d-MPH | |
| Tₘₐₓ (h) - median (min, max) | 6.00 | (4.0, 24.1) | 8.00 | (4.9, 30.0) | 6.50 | (1.0, 8.0) | 4.00 | (1.5, 7.0) |
| Cₘₐₓ (ng/mL) - % CV (Geo Mean) | 22.6 | (5.91) | 36.2 | (12.9) | 33.8 | (26.2) | 23.4 | (27.0) |
| AUC₀₋₂₄ (h*ng/mL) - % CV (Geo | 31.2 | (85.5) | 34.4 | (199) | 31.1 | (210) | 24.8 | (212) |
| Mean) | | | | | | | | |
| AUC_{inf} (h*ng/mL) - % CV (Geo | 32.1 | (132) | 41.0 | (306) | 31.9 | (216) | 25.6 | (217) |
| Mean) | | | | | | | | |
| CL/F (L/h) - % CV (Geo Mean) | 32.6 | (283) | 41.3 | (304) | 30.9 | (161) | 25.1 | (159) |

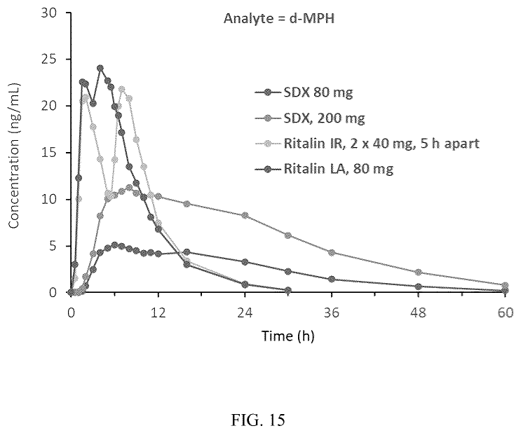

FIG. 15 summarizes the data in Table 5. The PK profile for both the single 80 mg and single 200 mg doses of serdexmethylphenidate both show lower Cₘₐₓ concentrations with a longer Tₘₐₓ compared to Ritalin. This results in higher AUC₀₋₂₄ and AUC_{inf} compared to Ritalin. This suggests that serdexmethylphenidate will effectively treat IH.

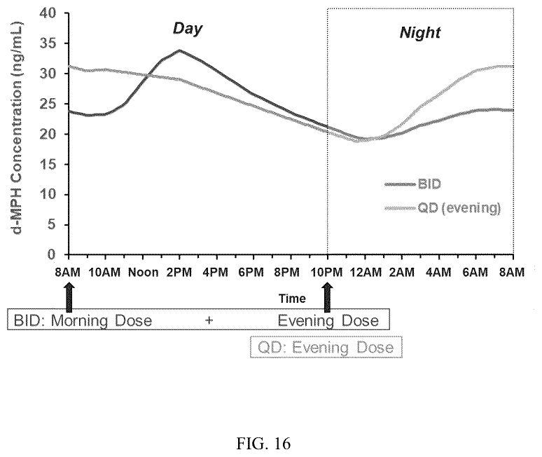

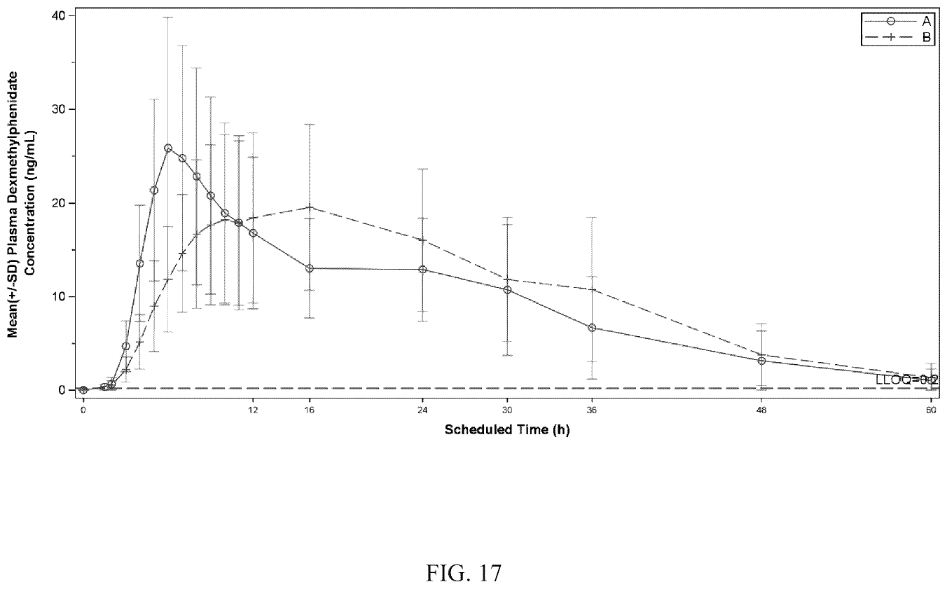

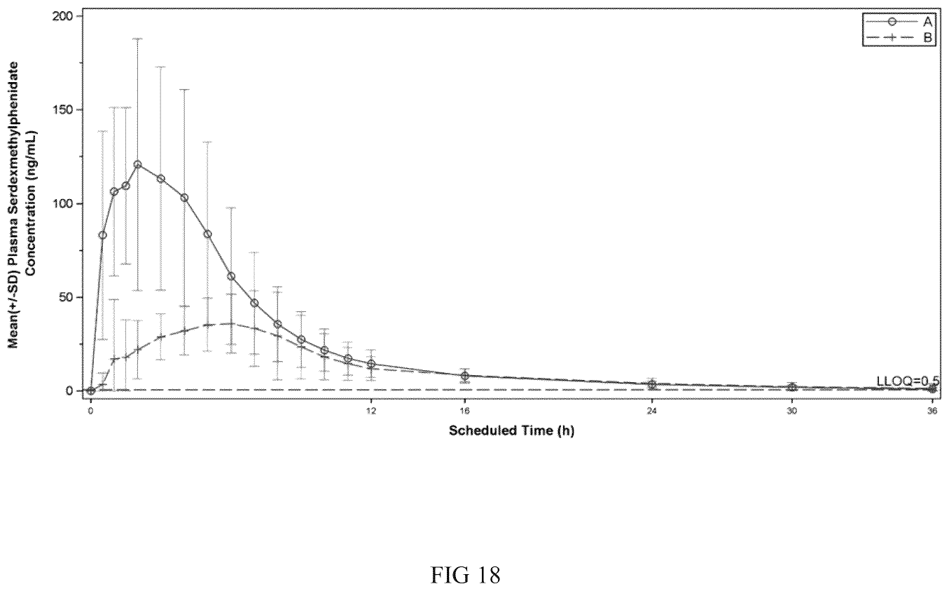

FIG. 16 shows d-MPH plasma concentrations following a single 240 mg serdexmethylphenidate dose administered at 10 PM (before bed) compared to two 120 mg serdexmethylphenidate doses administered at 10 PM (before bed) and mid-day. FIG. 17 shows plasma concentrations and standard deviations of d-methylphenidate following administering 240 mg of serdexmethylphenidate once daily in the evening (B) compared to once daily in the morning (A). FIG. 18 shows plasma serdexmethylphenidate concentrations and standard deviations for 240 mg of serdexmethylphenidate administered once daily in the evening (B) compared to once daily in the morning (A).

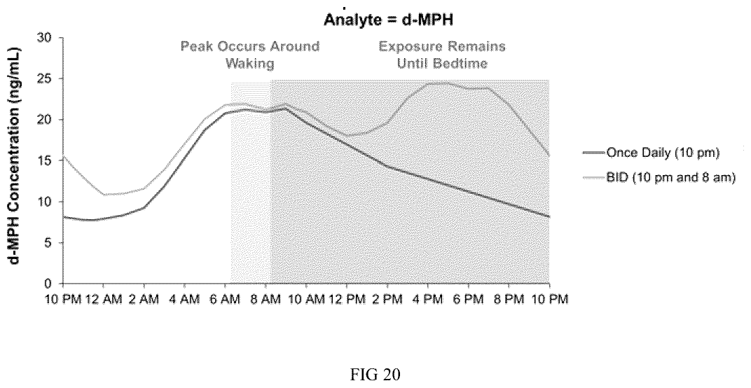

FIG. 20 shows similar PK data for plasma d-MPH levels to the data from FIG. 16. Both support that a single nighttime dose of serdexmethylphenidate provides maximum plasma d-MPH levels shortly after waking.

The data presented in the preceding examples supports the conclusion that serdexmethylphenidate has several advantages over other treatments for IH. These are summarized in Table 6.

TABLE 6
Comparison of serdexmethylphenidate to other
prescription treatments for sleep disorders

| Key | Serdex- | | | | Wake |
|---|---|---|---|---|---|
| Attribute | methylphenidate | XYWAV ® | LUMRYZ ® | WAKIX ® | Promoters |
| IH Indication | A | A | | | |
| EDS | A | A | A | M | A |
| Sleep Inertia | A | M | | M | |
| Brain Fog | A | | | | |
| Dosing | A | M | A | A | A |
| Schedule | M | D | D | A | M |
| REM Sleep | A | D | D | A | A |
| Salt | A | M | D | A | A |
| Restricted | | | | | |
| Diet | | | | | |

A—Advantage;
M—Mixed;
D—Disadvantage;
Blank—Gap

Table 6 shows that amongst the current treatments for sleep disorders, serdexmethylphenidate potentially provides the most patient benefits. Most importantly, serdexmethylphenidate improves IH, EDS, SI, and BF symptoms; none of the other treatment options in Table 6 improve symptoms in all of the instant sleep disorder. Furthermore, serdexmethylphenidate does not result in abnormal Rapid Eye Movement (REM) sleep cycles; whereas both XYWAV® and LUMRYZ® list abnormal REM sleep as a adverse event. Additionally, serdexmethylphenidate has advantageous dosing amounts and dosing schedules compared to both XYWAV® and LUMRYZ®. For example, XYWAV® requires two nighttime dosages, whereas serdexmethylphenidate reduces sleep disorder symptoms with only a single dose before bedtime.

Alternatively, in certain aspects, the present technology relates to a method of treating Idiopathic Hypersomnia (IH), in a patient in need thereof, the method comprising administering to the patient in need thereof a pharmaceutical composition comprising a therapeutically effective amount of the of a conjugate of d-methylphenidate having the structure of Formula I.

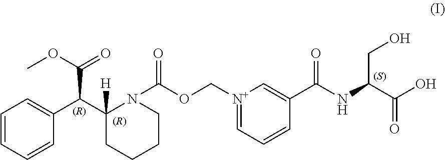

or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime. Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to a method of treating Idiopathic Hypersomnia associated with at least one of Excessive Daytime Sleepiness, Sleep Inertia, and Brain Fog, in a patient in need thereof, the method comprising administering to the patient in need thereof a pharmaceutical composition comprising a therapeutically effective amount of the of a conjugate of d-methylphenidate having the structure of Formula I, or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime. Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to a method of treating Excessive Daytime Sleepiness (EDS) in a patient in need thereof, the method comprising administering to the patient in need thereof a pharmaceutical composition comprising a therapeutically effective amount of the of a conjugate of d-methylphenidate having the structure of Formula I or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime. Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to a method of treating Excessive Daytime Sleepiness associated with at least one of Idiopathic Hypersomnia, Sleep Inertia, and Brain Fog, in a patient in need thereof, the method comprising administering to the patient in need thereof a pharmaceutical composition comprising a therapeutically effective amount of the of a conjugate of d-methylphenidate having the structure of Formula I, or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime. Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to a method of treating Sleep Inertia (SI) in a patient in need thereof, the method comprising administering to the patient in need thereof a pharmaceutical composition comprising a therapeutically effective amount of the of a conjugate of d-methylphenidate having the structure of Formula I or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime . . . . Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to a method of treating Sleep Inertia (SI) associated with at least one of Excessive Daytime Sleepiness, Idiopathic Hypersomnia, and Brain Fog, in a patient in need thereof, the method comprising administering to the patient in need thereof a pharmaceutical composition comprising a therapeutically effective amount of the of a conjugate of d-methylphenidate having the structure of Formula I, or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime . . . . Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to a method of treating Brain Fog (BF) in a patient in need thereof, the method comprising administering to the patient in need thereof a pharmaceutical composition comprising a therapeutically effective amount of the of a conjugate of d-methylphenidate having the structure of Formula I or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime . . . . Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to a method of treating Brain Fog (BF) associated with at least one of Excessive Daytime Sleepiness, Idiopathic Hypersomnia, and Idiopathic Hypersomnia, in a patient in need thereof, the method comprising administering to the patient in need thereof a pharmaceutical composition comprising a therapeutically effective amount of the of a conjugate of d-methylphenidate having the structure of Formula I, or a pharmaceutically acceptable salt thereof, wherein the pharmaceutical composition comprising a conjugate of d-methylphenidate is administered once to twice a day. Alternatively, in certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime . . . . Alternatively, the composition comprising the conjugate of d-methylphenidate having a structure of formula I is administered in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

In certain aspects, the present technology relates to methods of treating IH, EDS, SI, and/or BF in patients suffering from another disease and/or condition where IH, EDS, SI, and/or BF are symptomatic of the disease and/or condition. In certain aspects, the pharmaceutical composition comprising a conjugate of d-methylphenidate is administered once to twice a day, with the first dose administered during the daytime and the second dose about 0-3 hours before bedtime. In other certain aspects, the pharmaceutical composition comprising a conjugate of d-methylphenidate is administered once a day about 0-3 hours before bedtime. Alternatively, in certain aspects the pharmaceutical composition comprising a conjugate of d-methylphenidate is administered once in a single dose about 0-3 hours before bedtime. Alternatively, the composition comprising the conjugate of d-methylphenidate having a structure of formula I is administered once a day in the evening or before the patient wishes to fall asleep about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime. Alternatively, the conjugate of d-methylphenidate or pharmaceutically acceptable salt thereof is administered once per day. The therapeutically effective amount of the conjugate of d-methylphenidate or pharmaceutically acceptable salt thereof to be administered is about 50 mg to about 500 mg and alternatively is selected from the group consisting of about 80 mg, about 160 mg, about 240 mg, and about 320 mg.

1. A method of treating Idiopathic Hypersomnia in a patient, the method comprising: administering to a patient in need thereof before bedtime composition comprising a therapeutically effective amount of a composition comprising a conjugate of d-methylphenidate compound having a structure of Formula I:

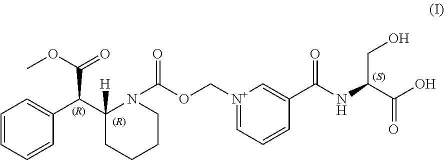

or a pharmaceutically acceptable salt thereof.

2. The method of 1, wherein the patient is suffering from at least one of Excessive Daytime Sleepiness (EDS), Sleep Inertia (SI) or Brain Fog (BF).

3. The method of 1 or 2, wherein the composition comprising the conjugate of d-methylphenidate having a structure of formula I is administered once a day or twice a day.

4. The method of 3, wherein the composition comprising the conjugate of d-methylphenidate having a structure of formula I is administered once a day.

5. The method of 3-4, wherein the therapeutically effective amount of the conjugate of d-methylphenidate or pharmaceutically acceptable salt thereof ranges from about 50 to about 350 mg.

6. The method of 5, wherein the therapeutically effective amount of the conjugate of d-methylphenidate or pharmaceutically acceptable salt thereof is selected from the group consisting of about 80 mg, about 160 mg, about 240 mg, and about 320 mg.

7. The method of 5 or 6, wherein composition comprising the conjugate of d-methylphenidate having a structure of formula I is administered in the evening or before bedtime, alternatively about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime.

8. A method of treating Excessive Daytime Sleepiness (EDS) in a patient, the method comprising: administering to a patient in need thereof a therapeutically effective amount of a composition comprising a conjugate of d-methylphenidate compound having a structure of Formula I:

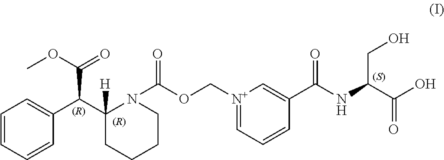

or a pharmaceutically acceptable salt thereof.

9. The method of 8, wherein the patient is suffering from at least one of Idiopathic Hypersomnia (IH), Sleep Inertia (SI) or Brain Fog (BF).

10. The method of 8 or 9, wherein the composition comprising the conjugate of d-methylphenidate having a structure of formula I is administered once a day or twice a day.

11. The method of 10, wherein the composition comprising the conjugate of d-methylphenidate having a structure of formula I is administered once a day.

12. The method of 10-11, wherein the therapeutically effective amount of the conjugate of d-methylphenidate or pharmaceutically acceptable salt thereof ranges from about 50 to about 350 mg.

13. The method of 12, wherein the therapeutically effective amount of the conjugate of d-methylphenidate or pharmaceutically acceptable salt thereof is selected from the group consisting of about 80 mg, about 160 mg, about 240 mg, and about 320 mg.

14. The method of 12 or 13, wherein composition comprising the conjugate of d-methylphenidate having a structure of formula I is administered in the evening or before bedtime, alternatively about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime.

15. A method of treating Sleep Inertia (SI) in a patient, the method comprising: administering to a patient in need thereof a therapeutically effective amount of a composition comprising a conjugate of d-methylphenidate compound having a structure of Formula I:

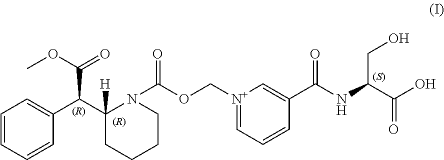

or a pharmaceutically acceptable salt thereof.

16. The method of 15, wherein the patient is suffering from at least one of Excessive Daytime Sleepiness (EDS), Idiopathic Hypersomnia (IH) or Brain Fog (BF).

17. The method of 15 or 16, wherein the composition comprising the conjugate of d-methylphenidate having a structure of formula I is administered once a day or twice a day.

18. The method of 17, wherein the composition comprising the conjugate of d-methylphenidate having a structure of formula I is administered once a day.

19. The method of 17-18, wherein the therapeutically effective amount of the conjugate of d-methylphenidate or pharmaceutically acceptable salt thereof ranges from about 50 to about 350 mg.

20. The method of 19, wherein the therapeutically effective amount of the conjugate of d-methylphenidate or pharmaceutically acceptable salt thereof is selected from the group consisting of about 80 mg, about 160 mg, about 240 mg, and about 320 mg.

21. The method of 19 or 20, wherein composition comprising the conjugate of d-methylphenidate having a structure of formula I is administered in the evening or before bedtime, alternatively about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime.

22. A method of treating Brain Fog (BF) in a patient, the method comprising: administering to a patient in need thereof a therapeutically effective amount of a composition comprising a conjugate of d-methylphenidate compound having a structure of Formula I:

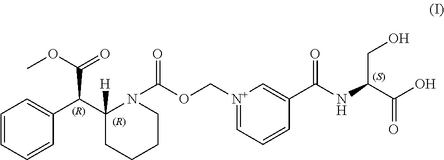

or a pharmaceutically acceptable salt thereof.

23. The method of 22, wherein the patient is suffering from at least one of Excessive Daytime Sleepiness (EDS), Sleep Inertia (SI) or Idiopathic Hypersomnia (IH).

24. The method of 22 or 23, wherein the composition comprising the conjugate of d-methylphenidate having a structure of formula I is administered once a day or twice a day.

25. The method of 24, wherein the composition comprising the conjugate of d-methylphenidate having a structure of formula I is administered once a day.

26. The method of 24-25, wherein the therapeutically effective amount of the conjugate of d-methylphenidate or pharmaceutically acceptable salt thereof ranges from about 50 to about 350 mg.

27. The method of 26, wherein the therapeutically effective amount of the conjugate of d-methylphenidate or pharmaceutically acceptable salt thereof is selected from the group consisting of about 80 mg, about 160 mg, about 240 mg, and about 320 mg.

28. The method of 26 or 27, wherein composition comprising the conjugate of d-methylphenidate having a structure of formula I is administered in the evening or before bedtime, alternatively about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, alternatively immediately before bedtime.

Alternatively, certain aspects of the presently described technology include a method of treating idiopathic hypersomnia in a patient suffering therefrom, or optionally in addition suffering from at least one of Excessive Daytime Sleepiness (EDS), Brain Fog (BF) and/or Sleep Inertia (SI), the method comprising: administering to a patient in need thereof a therapeutically effective amount of a composition comprising a conjugate of d-methylphenidate compound having a structure of Formula I or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of the conjugate of d-methylphenidate of Formula I or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of the conjugate of d-methylphenidate of Formula I or pharmaceutically acceptable salt is selected from the group consisting of about 80 mg, about 160 mg, about 240 mg, and about 320 mg.

Alternatively, certain aspects of the presently described technology include a method of treating Excessive Daytime Sleepiness in a patient suffering therefrom, or optionally in addition suffering from at least one of Idiopathic Hypersomnia (IH), Brain Fog (BF) and/or Sleep Inertia (SI), the method comprising: administering to a patient in need thereof a therapeutically effective amount of a composition comprising a conjugate of d-methylphenidate compound having a structure of Formula I or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of the conjugate of d-methylphenidate of Formula I or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of the conjugate of d-methylphenidate of Formula I or pharmaceutically acceptable salt is selected from the group consisting of about 80 mg, about 160 mg, about 240 mg, and about 320 mg.

Alternatively, certain aspects of the presently described technology include a method of treating Brain Fog in a patient suffering therefrom, or optionally in addition suffering from at least one of Idiopathic Hypersomnia (IH), Excessive Daytime Sleepiness (EDS) and/or Sleep Inertia (SI), the method comprising: administering to a patient in need thereof a therapeutically effective amount of a composition comprising a conjugate of d-methylphenidate compound having a structure of Formula I or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of the conjugate of d-methylphenidate of Formula I or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of the conjugate of d-methylphenidate of Formula I or pharmaceutically acceptable salt is selected from the group consisting of about 80 mg, about 160 mg, about 240 mg, and about 320 mg.

Alternatively, certain aspects of the presently described technology include a method of treating Sleep Inertia in a patient suffering therefrom, or optionally in addition suffering from at least one of Idiopathic Hypersomnia (IH), Excessive Daytime Sleepiness (EDS) and/or Brain Fog (BF), the method comprising: administering to a patient in need thereof a therapeutically effective amount of a composition comprising a conjugate of d-methylphenidate compound having a structure of Formula I or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of the conjugate of d-methylphenidate of Formula I or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of the conjugate of d-methylphenidate of Formula I or pharmaceutically acceptable salt is selected from the group consisting of about 80 mg, about 160 mg, about 240 mg, and about 320 mg.

Alternatively, certain aspects of the presently described technology include a method of treating idiopathic hypersomnia in a patient suffering therefrom, or optionally in addition suffering from at least one of Excessive Daytime Sleepiness (EDS), Brain Fog (BF) and/or Sleep Inertia (SI), the method comprising: administering to a patient in need thereof a therapeutically effective amount serdexmethylphenidate or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of serdexmethylphenidate or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of serdexmethylphenidate or pharmaceutically acceptable salt is selected from the group consisting of about 80 mg, about 160 mg, about 240 mg, and about 320 mg.

Alternatively, certain aspects of the presently described technology include a method of treating Excessive Daytime Sleepiness in a patient suffering therefrom, or optionally in addition suffering from at least one of Idiopathic Hypersomnia (IH), Brain Fog (BF) and/or Sleep Inertia (SI), the method comprising: administering to a patient in need thereof a therapeutically effective amount of a composition comprising serdexmethylphenidate or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of serdexmethylphenidate or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of serdexmethylphenidate or pharmaceutically acceptable salt is selected from the group consisting of about 80 mg, about 160 mg, about 240 mg, and about 320 mg.

Alternatively, certain aspects of the presently described technology include a method of treating Brain Fog in a patient suffering therefrom, or optionally in addition suffering from at least one of Idiopathic Hypersomnia (IH), Excessive Daytime Sleepiness (EDS) and/or Sleep Inertia (SI), the method comprising: administering to a patient in need thereof a therapeutically effective amount of a composition comprising serdexmethylphenidate or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of serdexmethylphenidate or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of serdexmethylphenidate or pharmaceutically acceptable salt is selected from the group consisting of about 80 mg, about 160 mg, about 240 mg, and about 320 mg.

Alternatively, certain aspects of the presently described technology include a method of treating Sleep Inertia in a patient suffering therefrom, or optionally in addition suffering from at least one of Idiopathic Hypersomnia (IH), Excessive Daytime Sleepiness (EDS) and/or Brain Fog (BF), the method comprising: administering to a patient in need thereof a therapeutically effective amount of a composition comprising serdexmethylphenidate or a pharmaceutically acceptable salt thereof. The composition may be administered once or twice a day. If the composition is administered once a day, the time of the administration should in the evening or optionally at about 0-3 hours before bedtime, alternatively 0-2 hours before bedtime, alternatively 0-90 minutes before bedtime, alternatively 0-60 minutes before bedtime, alternatively 0-45 minutes before bedtime, alternatively 0-30 minutes before bedtime, alternatively 0-20 minutes before bedtime, alternatively 0-15 minutes before bedtime, alternatively 0-10 minutes before bedtime, alternatively 0-5 minutes before bedtime, or alternatively immediately before bedtime. The therapeutically effective amount of serdexmethylphenidate or pharmaceutically acceptable salt thereof ranges from about 50 to about 350 mg, and optionally may be 60-340, 70 to 330, 80 to 320, 90 to 310 and 100 to about 300. Alternatively, the therapeutically effective amount of the conjugate of serdexmethylphenidate or pharmaceutically acceptable salt is selected from the group consisting of about 80 mg, about 160 mg, about 240 mg, and about 320 mg.

The presently described technology is now described in such full, clear, concise and exact terms as to enable any person skilled in the art to which it pertains, to practice the same. It is to be understood that the foregoing describes preferred embodiments of the technology and that modifications may be made therein without departing from the spirit or scope of the invention as set forth in the appended claims.

1. A method of treating Idiopathic Hypersomnia in a patient, the method comprising:
administering to the patient in need thereof before bedtime a composition comprising a therapeutically effective amount of a conjugate of d-methylphenidate having a structure of Formula I:

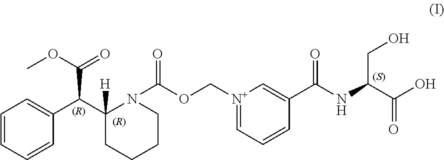

or a pharmaceutically acceptable salt thereof,
wherein the composition comprising the conjugate of d-methylphenidate is administered about 0-3 hours before the bedtime of the patient.

2. The method of 1, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

3. The method of 1 or 2, wherein the conjugate of d-methylphenidate is serdexmethylphenidate chloride.

4. The method of 1-3, wherein the therapeutically effective amount of the conjugate of d-methylphenidate is between 0.1 mg and 500 mg per day.

5. The method of 4, wherein the therapeutically effective amount of the conjugate f d-methylphenidate is between 50 mg and 350 mg per day.

6. The method of 5, wherein the therapeutically effective amount of the conjugate f d-methylphenidate is selected from the group consisting of 80, 160, 240, or 320 mg per day

7. The method of 6, wherein the therapeutically effective amount of the conjugate of d-methylphenidate is 240 mg per day.

8. The method of 1-5, wherein the therapeutically effective amount of a composition comprising serdexmethylphenidate chloride, wherein the therapeutically effective amount is selected from 87.5, 171.3, 257.0, or 342.7 mg per day.

9. The method of 1-8, wherein the composition comprising the conjugate of d-methylphenidate is administered once per day (QD).

10. The method of 1-9, wherein the Idiopathic Hypersomnia is associated with at least one additional sleep disorder selected from the group consisting of Excessive Daytime Sleepiness, Sleep Inertia, and Brain Fog.

11. The method of 1-10, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

12. The method of 11, wherein the decrease in at least one sleep disorder test score is an at least 10% decrease in the Idiopathic Hypersomnia Severity Scale score.

13. The method of 12, wherein the at least 10% decrease in the Idiopathic Hypersomnia Severity scale is an at least 4 point decrease in the Idiopathic Hypersomnia Scale score.

14. The method of 13, wherein the at least 10% decrease in the Idiopathic Hypersomnia Severity Scale score is an at least 10 point decrease in the Idiopathic Hypersomnia Severity Scale score.

15. The method of 11-14, wherein the decrease is measured at least 1 week after an initial dosage.

16. The method of 11 wherein the 10% decrease results in lowering the level of Idiopathic Hypersomnia severity.

17. The method of 6, wherein the patient in need thereof has a Idiopathic Hypersomnia Severity scale score of greater than 22 prior to starting the treatment.

18. The method of 17, wherein the decrease in at least one sleep disorder test score is an at least 10% decrease in the Epworth Sleepiness Scale score.

19. The method of 18, wherein the at least 10% decrease in the Epworth Sleepiness Scale score is an at least 2-point decrease in the Epworth Sleepiness Scale score.

20. The method of 19, wherein the at least 10% decrease in the Epworth Sleepiness Scale score is an at least 6 point decrease in the Epworth Sleepiness Scale score.

21. The method of 16-20, wherein the patient in need thereof has an Epworth Sleepiness Scale score of greater than 10 prior to starting the treatment.

22. The method of 16-21, wherein the decrease is measured at least 1 week after an initial dosage.

23. The method of any one of 1-22, wherein the composition is an oral dosage formulation.

24. The method of 23, wherein the oral dosage formulation is a liquid dosage formulation selected from the group consisting of syrups, emulsions or suspensions.

25. The method of 23, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

26. The method of 23, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

27. The method of 1-23, wherein the composition comprises at least one additional active pharmaceutical ingredient.

28. The method of 27, wherein the at least one additional active pharmaceutical ingredient is a sleep aid.

29. A method of treating Excessive Daytime Sleepiness in a patient, the method comprising:
administering to the patient in need thereof before bedtime a composition comprising a therapeutically effective amount of a conjugate of d-methylphenidate having a structure of Formula I:

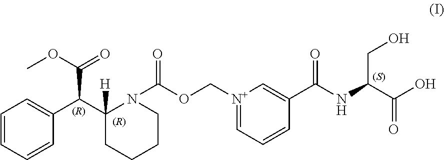

or a pharmaceutically acceptable salt thereof,
wherein the composition comprising a conjugate of d-methylphenidate is administered 0-3 hours before the bedtime of the patient.

30. The method of 29, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

31. The method of 29 or 30, wherein the conjugate of d-methylphenidate is serdexmethylphenidate chloride.

32. The method of 29-31, wherein the therapeutically effective amount of the conjugate of d-methylphenidate is between 0.1 mg and 500 mg per day.

33. The method of 32, wherein the therapeutically effective amount of the conjugate f d-methylphenidate is between 50 mg and 350 mg per day.

34. The method of 33, wherein the therapeutically effective amount of the conjugate f d-methylphenidate is selected from the group consisting of 80, 160, 240, or 320 mg per day

35. The method of 34, wherein the therapeutically effective amount of the conjugate of d-methylphenidate is 240 mg per day.

36. The method of 29-33, wherein the therapeutically effective amount of a composition comprising serdexmethylphenidate chloride, wherein the therapeutically effective amount is selected from 87.5, 171.3, 257.0, or 342.7 mg per day.

37. The method of 29-36, wherein the composition comprising the conjugate of d-methylphenidate is administered once per day (QD).

38. The method of 29-37, wherein the Excessive Daytime Sleepiness is associated with at least one additional sleep disorder selected from the group consisting of Idiopathic Hypersomnia, Sleep Inertia, and Brain Fog.

39. The method of 29-38, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

40. The method of 39, wherein the decrease in at least one sleep disorder test score is an at least 10% decrease in the Epworth Sleepiness Scale score.

41. The method of 40, wherein the decrease in at least one sleep disorder test score is an at least 10% decrease in the Epworth Sleepiness Scale score.

42. The method of 41, wherein the at least 10% decrease in the Epworth Sleepiness Scale score is an at least 2-point decrease in the Epworth Sleepiness Scale score.

43. The method of 42, wherein the at least 10% decrease in the Epworth Sleepiness Scale score is an at least 6 point decrease in the Epworth Sleepiness Scale score.

44. The method of 39-43, wherein the patient in need thereof has an Epworth Sleepiness Scale score of greater than 10 prior to starting the treatment.

45. The method of 39-44, wherein the decrease is measured at least 1 week after an initial dosage.

46. The method of any one of 29-45, wherein the composition is an oral dosage formulation.

47. The method of 46, wherein the oral dosage formulation is a liquid dosage formulation selected from the group consisting of syrups, emulsions or suspensions.

48. The method of 46, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

49. The method of 46, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

50. The method of 29-49, wherein the composition comprises at least one additional active pharmaceutical ingredient.

51. The method of 50, wherein the at least one additional active pharmaceutical ingredient is a sleep aid.

52. A method of treating Sleep Inertia in a patient, the method comprising:
administering to the patient in need thereof before bedtime a composition comprising a therapeutically effective amount of a conjugate of d-methylphenidate having a structure of Formula I:

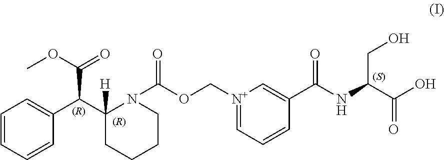

or a pharmaceutically acceptable salt thereof,
wherein the composition comprising a conjugate of d-methylphenidate is administered 0-3 hours before the bedtime of the patient.

53. The method of 52, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

54. The method of 52 or 53, wherein the conjugate of d-methylphenidate is serdexmethylphenidate chloride.

55. The method of 52-54, wherein the therapeutically effective amount of the conjugate of d-methylphenidate is between 0.1 mg and 500 mg per day.

56. The method of 55, wherein the therapeutically effective amount of the conjugate f d-methylphenidate is between 50 mg and 350 mg per day.

57. The method of 56, wherein the therapeutically effective amount of the conjugate f d-methylphenidate is selected from the group consisting of 80, 160, 240, or 320 mg per day

58. The method of 57, wherein the therapeutically effective amount of the conjugate of d-methylphenidate is 240 mg per day.

59. The method of 52-56, wherein the therapeutically effective amount of a composition comprising serdexmethylphenidate chloride, wherein the therapeutically effective amount is selected from 87.5, 171.3, 257.0, or 342.7 mg per day.

60. The method of 52-61, wherein the composition comprising the conjugate of d-methylphenidate is administered once per day (QD).

61. The method of 52-60, wherein the Sleep Inertia is associated with at least one additional sleep disorder selected from the group consisting of Excessive Daytime Sleepiness, Idiopathic Hypersomnia, and Brain Fog.

62. The method of 52-61, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

63. The method of 62, wherein the decrease in at least one sleep disorder test score is an at least 10% decrease in the Sleep Inertia Visual Analog Scale (SI-VAS)score.

64. The method of 63, wherein the decrease in at least one sleep disorder test score is an at least 10% decrease in the Sleep Inertia Visual Analog Scale (SI-VAS)score.

65. The method of 64, wherein the at least 10% decrease in the Epworth Sleepiness Scale score is an at least 5-point decrease in the Sleep Inertia Visual Analog Scale (SI-VAS)score.

66. The method of 65, wherein the at least 10% decrease in the Epworth Sleepiness Scale score is an at least 15 point decrease in the Sleep Inertia Visual Analog Scale (SI-VAS)score.

67. The method of 62-66, wherein the patient in need thereof has an Sleep Inertia Visual Analog Scale (SI-VAS) score of greater than 50 prior to starting the treatment.

68. The method of 62-67, wherein the decrease is measured at least 1 week after an initial dosage.

69. The method of any one of 52-68, wherein the composition is an oral dosage formulation.

70. The method of 69, wherein the oral dosage formulation is a liquid dosage formulation selected from the group consisting of syrups, emulsions or suspensions.

71. The method of 69, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

72. The method of 69, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

73 The method of 52-73, wherein the composition comprises at least one additional active pharmaceutical ingredient.

74. The method of 73, wherein the at least one additional active pharmaceutical ingredient is a sleep aid.

75. A method of treating Brain Fog in a patient, the method comprising:
administering to the patient in need thereof before bedtime a composition comprising a therapeutically effective amount of a conjugate of d-methylphenidate having a structure of Formula I:

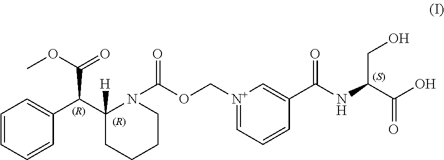

or a pharmaceutically acceptable salt thereof,
wherein the composition comprising a conjugate of d-methylphenidate is administered 0-3 hours before the bedtime of the patient.

76. The method of 75, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

77. The method of 75 or 76, wherein the conjugate of d-methylphenidate is serdexmethylphenidate chloride.

78. The method of 75-78, wherein the therapeutically effective amount of the conjugate of d-methylphenidate is between 0.1 mg and 500 mg per day.

79. The method of 78, wherein the therapeutically effective amount of the conjugate f d-methylphenidate is between 50 mg and 350 mg per day.

80. The method of 79, wherein the therapeutically effective amount of the conjugate f d-methylphenidate is selected from the group consisting of 80, 160, 240, or 320 mg per day

81. The method of 80, wherein the therapeutically effective amount of the conjugate of d-methylphenidate is 240 mg per day.

82. The method of 75-79, wherein the therapeutically effective amount of a composition comprising serdexmethylphenidate chloride, wherein the therapeutically effective amount is selected from 87.5, 171.3, 257.0, or 342.7 mg per day.

83. The method of 75-82, wherein the composition comprising the conjugate of d-methylphenidate is administered once per day (QD).

84. The method of 75-83, wherein the Brin Fog is associated with at least one additional sleep disorder selected from the group consisting of Excessive Daytime Sleepiness, Sleep Inertia, and Idiopathic Hypersomnia.

85. The method of 75-84, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

86. The method of 85, wherein the decrease in at least one sleep disorder test score is an at least 10% decrease in the Brain Fog Symptom Scale score.

87. The method of 86, wherein the at least 10% decrease in the Idiopathic Hypersomnia Severity scale is an at least 5 point decrease in the Brain Fog Symptom Scale score.

88. The method of 87, wherein the at least 10% decrease in the Idiopathic Hypersomnia Severity Scale score is an at least 20 point decrease in the I Brain Fog Symptom Scale score.

89. The method of 85-88, wherein the decrease is measured at least 1 week after an initial dosage.

90. The method of 85-89 wherein the 10% decrease results in lowering the level of Brain Fog severity.

91. The method of 85-90, wherein the patient in need thereof has a Brain Fog Symptom Scale score of greater than 35 prior to starting the treatment.

92. The method of any one of 75-91, wherein the composition is an oral dosage formulation.

93. The method of 92, wherein the oral dosage formulation is a liquid dosage formulation selected from the group consisting of syrups, emulsions or suspensions.

94. The method of 92, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

95. The method of 92, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

96. The method of 75-96, wherein the composition comprises at least one additional active pharmaceutical ingredient.

97. The method of 96, wherein the at least one additional active pharmaceutical ingredient is a sleep aid.

98. A method of treating Idiopathic Hypersomnia in a patient, the method comprising:
administering to the patient in need thereof a composition comprising a therapeutically effective amount of a conjugate of d-methylphenidate having a structure of Formula I:

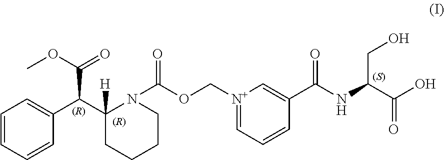

or a pharmaceutically acceptable salt thereof,
wherein the composition comprising the conjugate of d-methylphenidate is administered twice per day,
wherein the first dose is administered about 0 to 3 hours before bedtime and the second dose is administered about 8 to 12 hours after bedtime.

99. The method of 98, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

100. The method of 98 or 99, wherein the conjugate of d-methylphenidate is serdexmethylphenidate chloride.

101. The method of 98-100, wherein the therapeutically effective amount of the conjugate of d-methylphenidate is between 0.1 mg and 500 mg per day.

102. The method of 101, wherein the therapeutically effective amount of the conjugate f d-methylphenidate is between 50 mg and 350 mg per day.

103. The method of 102, wherein the therapeutically effective amount of the conjugate f d-methylphenidate is selected from the group consisting of 80, 160, 240, or 320 mg per day

104. The method of 103, wherein the therapeutically effective amount of the conjugate of d-methylphenidate is 240 mg per day.

105. The method of 98-104, wherein the therapeutically effective amount of a composition comprising serdexmethylphenidate chloride, wherein the therapeutically effective amount is selected from 87.5, 171.3, 257.0, or 342.7 mg per day.

106. The method of 98-105, wherein the Idiopathic Hypersomnia is associated with at least one additional sleep disorder selected from the group consisting of Excessive Daytime Sleepiness, Sleep Inertia, and Brain Fog.

107. The method of 98-106, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

108. The method of 107, wherein the decrease in at least one sleep disorder test score is an at least 10% decrease in the Idiopathic Hypersomnia Severity Scale score.

109. The method of 108, wherein the at least 10% decrease in the Idiopathic Hypersomnia Severity scale is an at least 4 point decrease in the Idiopathic Hypersomnia Scale score.

110. The method of 109, wherein the at least 10% decrease in the Idiopathic Hypersomnia Severity Scale score is an at least 10 point decrease in the Idiopathic Hypersomnia Severity Scale score.

111. The method of 107-110, wherein the decrease is measured at least 1 week after an initial dosage.

112. The method of 107-111 wherein the 10% decrease results in lowering the level of Idiopathic Hypersomnia severity.

113. The method of 112, wherein the patient in need thereof has a Idiopathic Hypersomnia Severity scale score of greater than 22 prior to starting the treatment.

114. The method of 107, wherein the decrease in at least one sleep disorder test score is an at least 10% decrease in the Epworth Sleepiness Scale score.

115. The method of 114, wherein the at least 10% decrease in the Epworth Sleepiness Scale score is an at least 2-point decrease in the Epworth Sleepiness Scale score.

116. The method of 115, wherein the at least 10% decrease in the Epworth Sleepiness Scale score is an at least 6 point decrease in the Epworth Sleepiness Scale score.

117. The method of 114-116, wherein the patient in need thereof has an Epworth Sleepiness Scale score of greater than 10 prior to starting the treatment.

118. The method of 114-117, wherein the decrease is measured at least 1 week after an initial dosage.

119. The method of any one of 98-118, wherein the composition is an oral dosage formulation.

120. The method of 119, wherein the oral dosage formulation is a liquid dosage formulation selected from the group consisting of syrups, emulsions or suspensions.

121. The method of 119, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

122. The method of 119, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

123. The method of 98-122, wherein the composition comprises at least one additional active pharmaceutical ingredient.

124. The method of 123, wherein the at least one additional active pharmaceutical ingredient is a sleep aid.

125. A method of treating Excessive Daytime Sleepiness in a patient, the method comprising:
administering to the patient in need thereof a composition comprising a therapeutically effective amount of a conjugate of d-methylphenidate having a structure of Formula I:

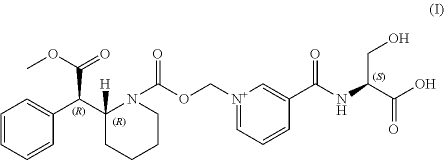

or a pharmaceutically acceptable salt thereof,
wherein the composition comprising the conjugate of d-methylphenidate is administered twice per day,
wherein the first dose is administered about 0 to 3 hours before bedtime and the second dose is administered about 8 to 12 hours after bedtime.

126. The method of 125, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

127. The method of 125 or 126, wherein the conjugate of d-methylphenidate is serdexmethylphenidate chloride

128. The method of 125-127, wherein the therapeutically effective amount of the conjugate of d-methylphenidate is between 0.1 mg and 500 mg per day.

129. The method of 128, wherein the therapeutically effective amount of the conjugate f d-methylphenidate is between 50 mg and 350 mg per day.

130. The method of 129, wherein the therapeutically effective amount of the conjugate f d-methylphenidate is selected from the group consisting of 80, 160, 240, or 320 mg per day

131. The method of 130, wherein the therapeutically effective amount of the conjugate of d-methylphenidate is 240 mg per day.

132. The method of 125-129, wherein the therapeutically effective amount of a composition comprising serdexmethylphenidate chloride, wherein the therapeutically effective amount is selected from 87.5, 171.3, 257.0, or 342.7 mg per day.

133. The method of 125-132, wherein the Excessive Daytime Sleepiness is associated with at least one additional sleep disorder selected from the group consisting of Idiopathic Hypersomnia, Sleep Inertia, and Brain Fog.

134. The method of 125-133, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

135. The method of 134, wherein the decrease in at least one sleep disorder test score is an at least 10% decrease in the Epworth Sleepiness Scale score.

136. The method of 135, wherein the decrease in at least one sleep disorder test score is an at least 10% decrease in the Epworth Sleepiness Scale score.

137. The method of 136, wherein the at least 10% decrease in the Epworth Sleepiness Scale score is an at least 2-point decrease in the Epworth Sleepiness Scale score.

138. The method of 137, wherein the at least 10% decrease in the Epworth Sleepiness Scale score is an at least 6 point decrease in the Epworth Sleepiness Scale score

139. The method of 134-138, wherein the patient in need thereof has an Epworth Sleepiness Scale score of greater than 10 prior to starting the treatment.

140. The method of 134-139, wherein the decrease is measured at least 1 week after an initial dosage.

141. The method of any one of 125-140, wherein the composition is an oral dosage formulation.

142. The method of 141, wherein the oral dosage formulation is a liquid dosage formulation selected from the group consisting of syrups, emulsions or suspensions.

143 The method of 141, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

144. The method of 141, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

145. The method of 125-144, wherein the composition comprises at least one additional active pharmaceutical ingredient.

146. The method of 146, wherein the at least one additional active pharmaceutical ingredient is a sleep aid.

147. A method of treating Sleep Inertia in a patient, the method comprising:
administering to the patient in need thereof a composition comprising a therapeutically effective amount of a conjugate of d-methylphenidate having a structure of Formula I:

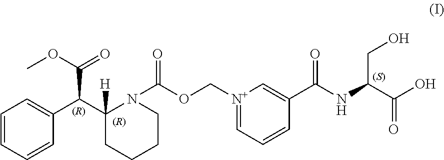

or a pharmaceutically acceptable salt thereof,
wherein composition comprising a conjugate of d-methylphenidate is administered 0-3 hours before bedtime.

148. The method of 147, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

149. The method of 147 or claim 148, wherein the conjugate of d-methylphenidate is serdexmethylphenidate chloride.

150. The method of 147-149, wherein the therapeutically effective amount of the conjugate of d-methylphenidate is between 0.1 mg and 500 mg per day.

151. The method of 150, wherein the therapeutically effective amount of the conjugate f d-methylphenidate is between 50 mg and 350 mg per day.

152. The method of 151, wherein the therapeutically effective amount of the conjugate f d-methylphenidate is selected from the group consisting of 80, 160, 240, or 320 mg per day

153. The method of 152, wherein the therapeutically effective amount of the conjugate of d-methylphenidate is 240 mg per day.

154. The method of 147-151, wherein the therapeutically effective amount of a composition comprising serdexmethylphenidate chloride, wherein the therapeutically effective amount is selected from 87.5, 171.3, 257.0, or 342.7 mg per day.

155. The method of 147-154, wherein the Sleep Inertia is associated with at least one additional sleep disorder selected from the group consisting of Excessive Daytime Sleepiness, Idiopathic Hypersomnia, and Brain Fog.

156. The method of 147-155, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

157. The method of 156, wherein the decrease in at least one sleep disorder test score is an at least 10% decrease in the Sleep Inertia Visual Analog Scale (SI-VAS)score.

158. The method of 157, wherein the decrease in at least one sleep disorder test score is an at least 10% decrease in the Sleep Inertia Visual Analog Scale (SI-VAS)score.

159. The method of 158, wherein the at least 10% decrease in the Sleep Inertia Visual Analog Scale (SI-VAS) score is an at least 5-point decrease in the Sleep Inertia Visual Analog Scale (SI-VAS) score.

160. The method of 157-159, wherein the at least 10% decrease in the Sleep Inertia Visual Analog Scale (SI-VAS) score is an at least 15 point decrease in the Sleep Inertia Visual Analog Scale (SI-VAS)score.

161. The method of 156-160, wherein the patient in need thereof has an Sleep Inertia Visual Analog Scale (SI-VAS) score of greater than 50 prior to starting the treatment.

162. The method of 156-161, wherein the decrease is measured at least 1 week after an initial dosage.

163. The method of any one of 147-162, wherein the composition is an oral dosage formulation.

164. The method of 163, wherein the oral dosage formulation is a liquid dosage formulation selected from the group consisting of syrups, emulsions or suspensions.

165. The method of 163, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

166. The method of 163, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

167. The method of 147-166, wherein the composition comprises at least one additional active pharmaceutical ingredient.

168. The method of 167, wherein the at least one additional active pharmaceutical ingredient is a sleep aid.

169. A method of treating Brain Fog in a patient, the method comprising:
administering to the patient in need thereof a composition comprising a therapeutically effective amount of a conjugate of d-methylphenidate having a structure of Formula I:

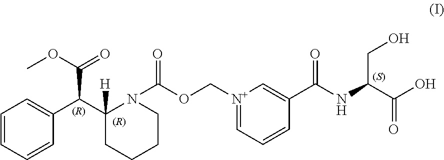

or a pharmaceutically acceptable salt thereof,
wherein composition comprising a conjugate of d-methylphenidate is administered 0-3 hours before bedtime.

170. The method of 169, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, i-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

171. The method of 169 or 170, wherein the conjugate of d-methylphenidate is serdexmethylphenidate chloride.

172. The method of 169-171, wherein the therapeutically effective amount of the conjugate of d-methylphenidate is between 0.1 mg and 500 mg per day.

173. The method of 172, wherein the therapeutically effective amount of the conjugate f d-methylphenidate is between 50 mg and 350 mg per day.

174. The method of 173, wherein the therapeutically effective amount of the conjugate f d-methylphenidate is selected from the group consisting of 80, 160, 240, or 320 mg per day

175. The method of 174, wherein the therapeutically effective amount of the conjugate of d-methylphenidate is 240 mg per day.

176. The method of 169-173, wherein the therapeutically effective amount of a composition comprising serdexmethylphenidate chloride, wherein the therapeutically effective amount is selected from 87.5, 171.3, 257.0, or 342.7 mg per day.

177. The method of 169-176, wherein the Brin Fog is associated with at least one additional sleep disorder selected from the group consisting of Excessive Daytime Sleepiness, Sleep Inertia, and Idiopathic Hypersomnia.

178. The method of 169-177, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

179. The method of 178, wherein the decrease in at least one sleep disorder test score is an at least 10% decrease in the Brain Fog Symptom Scale score.

180. The method of 179, wherein the at least 10% decrease in the Idiopathic Hypersomnia Severity scale is an at least 5 point decrease in the Brain Fog Symptom Scale score.

181. The method of 180, wherein the at least 10% decrease in the Idiopathic Hypersomnia Severity Scale score is an at least 20 point decrease in the I Brain Fog Symptom Scale score.

182. The method of 178-181, wherein the decrease is measured at least 1 week after an initial dosage.

183. The method of 178-182 wherein the 10% decrease results in lowering the level of Brain Fog severity.

184. The method of 178-183, wherein the patient in need thereof has a Brain Fog Symptom Scale score of greater than 35 prior to starting the treatment.

185. The method of any one of 169-184, wherein the composition is an oral dosage formulation.

186. The method of 185, wherein the oral dosage formulation is a liquid dosage formulation selected from the group consisting of syrups, emulsions or suspensions.

187. The method of 185, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

188. The method of 185, wherein the oral dosage formulation further comprises at least one excipient selected from the group consisting of antiadherents, binders, coatings, disintegrants, gel forming agents, fillers, flavors and colors, glidants, lubricants, preservatives, sorbents and sweeteners.

189. The method of 188, wherein the composition comprises at least one additional active pharmaceutical ingredient.

190. The method of claim 189, wherein the at least one additional active pharmaceutical ingredient is a sleep aid.

1. A method of treating Idiopathic Hypersomnia in a patient, the method comprising:
administering to the patient in need thereof before bedtime a composition comprising a therapeutically effective amount of a conjugate of d-methylphenidate having a structure of Formula I: 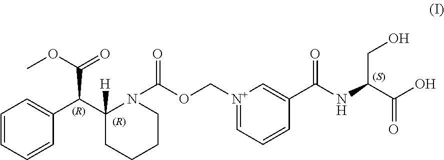
or a pharmaceutically acceptable salt thereof,
wherein the composition comprising the conjugate of d-methylphenidate is administered about 0-3 hours before the bedtime of the patient.

2. The method of claim 1, wherein the pharmaceutically acceptable salt is selected from the group consisting of acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, i-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, and undecylenate.

3. The method of claim 1, wherein the conjugate of d-methylphenidate is serdexmethylphenidate chloride.

4. The method of claim 1, wherein the therapeutically effective amount of the conjugate of d-methylphenidate is between 0.1 mg and 500 mg per day.

5. The method of claim 4, wherein the therapeutically effective amount of the conjugate f d-methylphenidate is between 50 mg and 350 mg per day.

6. The method of claim 5, wherein the therapeutically effective amount of the conjugate f d-methylphenidate is selected from the group consisting of 80, 160, 240, or 320 mg per day.

7. The method of claim 6, wherein the therapeutically effective amount of the conjugate of d-methylphenidate is 240 mg per day.

8. The method of claim 1, wherein the therapeutically effective amount of a composition comprising serdexmethylphenidate chloride, wherein the therapeutically effective amount is selected from 87.5, 171.3, 257.0, or 342.7 mg per day.

9. The method of claim 1, wherein the composition comprising the conjugate of d-methylphenidate is administered once per day (QD).

10. The method of claim 1, wherein the Idiopathic Hypersomnia is associated with at least one additional sleep disorder selected from the group consisting of Excessive Daytime Sleepiness, Sleep Inertia, and Brain Fog.

11. The method of claim 1, wherein administering the composition comprising the conjugate of d-methylphenidate results in a decrease in at least one sleep disorder test score selected from the group consisting of Epworth Sleepiness Scale (ESS), Idiopathic Hypersomnia Severity Scale (IHSS), Sleep Inertia Visual Analog Scale (SI-VAS), and Brain Fog Symptom Scale (BFS).

12. The method of claim 11, wherein the decrease in at least one sleep disorder test score is an at least 10% decrease in the Idiopathic Hypersomnia Severity Scale score.

13. The method of claim 12, wherein the at least 10% decrease in the Idiopathic Hypersomnia Severity scale is an at least 4 point decrease in the Idiopathic Hypersomnia Scale score.

14. (canceled)

15. (canceled)

16. The method of claim 11 wherein the 10% decrease results in lowering the level of Idiopathic Hypersomnia severity.

17. The method of claim 16, wherein the patient in need thereof has a Idiopathic Hypersomnia Severity scale score of greater than 22 prior to starting the treatment.

18-22. (canceled)

23. The method of claim 1, wherein the composition is an oral dosage formulation.

24. (canceled)

25. The method of claim 23, wherein the oral dosage formulation is a solid dosage formulation selected from the group consisting of a sublingual, a gummy, a chewable tablet, a rapidly dissolving tablet, a tablet, a capsule, a caplet, a troche, a lozenge, an oral powder, a thin strip, an oral thin film (OTF), and an oral strip.

26. (canceled)

27. The method of claim 1, wherein the composition comprises at least one additional active pharmaceutical ingredient.

28. (canceled)

29. A method of treating Excessive Daytime Sleepiness in a patient, the method comprising:
administering to the patient in need thereof before bedtime a composition comprising a therapeutically effective amount of a conjugate of d-methylphenidate having a structure of Formula I: 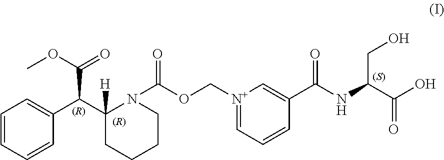
or a pharmaceutically acceptable salt thereof,
wherein the composition comprising the conjugate of d-methylphenidate is administered about 0-3 hours before the bedtime of the patient.

30.-51. (canceled)

52. A method of treating Sleep Inertia in a patient, the method comprising:
administering to the patient in need thereof before bedtime a composition comprising a therapeutically effective amount of a conjugate of d-methylphenidate having a structure of Formula I: 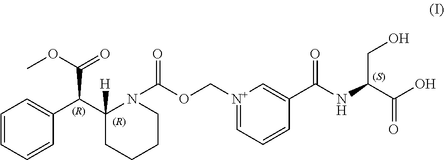
or a pharmaceutically acceptable salt thereof,
wherein the composition comprising the conjugate of d-methylphenidate is administered about 0-3 hours before the bedtime of the patient.

53-189. (canceled)
